(19) United States
(12) Patent Application Publication
(10) Pub. No.: US 2018/0344758 A1
(43) Pub. Date: Dec. 6, 2018

(54) Method of Treating Cancer with cGAMP or cGAsMP

(71) Applicant: LIPOGEN LLC, Mt. Laurel, NJ (US)

(72) Inventors: Pingwei Li, College Station, TX (US); Chenguang Wang, Mt. Laurel, NJ (US); Chang Shu, College Station, TX (US)

(73) Assignee: LIPOGEN LLC, Mt. Laurel, NJ (US)

(21) Appl. No.: 15/533,687

(22) PCT Filed: Dec. 15, 2015
(86) PCT No.: PCT/US15/65678

§ 371 Date: Jun. 7, 2017

Related U.S. Application Data
(60) Provisional application No. 62/093,221, filed on Dec. 17, 2014.

(51) Int. Cl. A61K 31/7084; A61P 35/00; A61P 35/02; C12P 19/36

(52) U.S. Cl. CPC A61K 31/7084; C12P 19/36; A61P 35/02; A61P 35/00

(57) ABSTRACT

In one embodiment, a method of treating cancer in a patient comprises administering cGAMP or cGAsMP to a patient having cancer and allowing the cGAMP or cGAsMP to treat the cancer. In another embodiment, a method for en2ymatically synthesizing and purifying cGAMP or cGAsMP comprises providing cGAS; combining cGAS with ATP or ATP phosphorothioate, respectively, and GTP to produce cGAMP or cGAsMP; separating cGAMP or cGAsMP from the cGAS and DNA by ultrafiltration; and purifying cGAMP or cGAsMP using ion exchange chromatography and optionally gel filtration chromatography.

### FIELD

A method of treating cancer with cGAMP or cGAsMP

### BACKGROUND

The cGAS-cGAMP-STING pathway has been discovered as part of the cell's innate immune responses to the presence of DNA in the cytoplasm of mammalian cells. A number of innate sensors for cytoplasmic DNA or RNA have been identified. See Barber G N, STING-dependent cytosolic DNA sensing pathways, Trends in immunology 35:88-93 (2014). Microbial DNA in the cytosol has long been known to induce potent innate immune responses by stimulating the expression of type I interferon. See Stetson D B, et al., Recognition of cytosolic DNA activates an IRF3-dependent innate immune response, Immunity 24:93-103 (2006). The search for cytosolic DNA sensors first lead to the discovery of STING (also known as MITA, ERIS, MPYS, and TMEM173), an adaptor protein located on the ER membrane that mediate the signaling to cytosolic DNA and bacterial cyclic dinucleotides such as c-di-GMP and c-di-AMP. FIG. 1; see also Ishikawa H, et al., STING is an endoplasmic reticulum adaptor that facilitates innate immune signalling, Nature 455:674-8 (2008). Although STING serves as a direct sensor of cyclic dinucleotides, it is not a direct sensor for cytosolic DNA and exhibits very low affinity for dsDNA. See Wu J, et al., Innate immune sensing and signaling of cytosolic nucleic acids, Annual review of immunology 32:461-88 (2014). In the search for cytosolic DNA sensor, Sun et. al. identified the enzyme cyclic GMP-AMP synthase (cGAS) as the cytosolic dsDNA sensor upstream of STING. Sun L, et al., Cyclic GMP-AMP synthase is a cytosolic DNA sensor that activates the type I interferon pathway, Science 339:786-91 (2013). cGAS is activated by dsDNA and catalyzes the synthesis of a noncanonical cyclic dinucleotide 2′,5′ cGAMP (referred to as cGAMP hereafter) from ATP and GTP. See Zhang X, et al., Cyclic GMP-AMP Containing Mixed Phosphodiester Linkages Is An Endogenous High-Affinity Ligand for STING, Molecular cell 51:226-35 (2013); see also FIG. 1.

cGAMP serves as an endogenous second messenger to stimulate the induction of type I interferons via STING. cGAMP binding by STING leads to the recruitment of the protein kinase TBK1 and transcription factor IRF3 to the signaling complex. See FIG. 1; see also Tanaka Y, et al., STING Specifies IRF3 Phosphorylation by TBK1 in the Cytosolic DNA Signaling Pathway, Science signaling 5:ra20 (2012).

Phosphorylation of IRF3 by TBK1 at the signaling complex promotes the oligomerization of IRF3 and its translocation into the nucleus where it activates the transcription of the IFN-β gene together with the transcription factor NF-κB. See Tanaka; FIG. 1.

The prior methods for synthesis of cGAMP used chemical synthesis methods, which included multiple steps and the use of various modified nucleotides. Gao P, et al., Structure-function analysis of STING activation by c[G(2′,5′)pA(3′,5′)p] and targeting by antiviral DMXAA, Cell 154:748-62 (2013).

The potential for cGAMP to treat cancer, however, has not been explored. This disclosure demonstrates the direct and potent tumor suppressive activity of cGAMP against certain tumor cell lines. This disclosure also provides a highly efficient protocol to synthesize cGAMP from ATP and GTP using recombinant human or mouse cGAS catalytic domain and an efficient technique to purify cGAMP.

### SUMMARY

In accordance with the description, a method of treating cancer in a patient comprises administering cGAMP or cGAsMP to a patient having cancer and allowing the cGAMP or cGAsMP to treat the cancer. In some embodiments, a method of inhibiting growth of cancer cells comprises providing a population of cancer cells; exposing the cancer cells to cGAMP or cGAsMP and allowing the cGAMP or cGAsMP to inhibit the growth of the cancer cells.

In some embodiments, STING expression level in the cancer is at least about 1, 1.2, 1.25, 1.3, 1.4, 1.5, 1.6, 1.7, 1.8, 1.9, 2.0, 2.25, 2.5, 2.75, 3.0, 3.25, 3.5, 3.75, 4.0, 4.25, or 4.5 fold higher than an average level in normal cells. In some embodiments, cGAS expression level are within the lower 50%, 45%, 40%, 35%, 30%, 25%, 20%, 15%, or 10% of patients, when evaluating the cGAS level in a pool of patients.

Additionally, in some aspects, a method for enzymatically synthesizing cGAMP comprises providing recombinant cGAS and combining cGAS with ATP, GTP, and dsDNA to synthesize cGAMP.

In some instances, no modified nucleotides are used in the synthesis method, synthesis may be conducted in a single pot, and/or synthesis may be conducted in a single step.

In some aspects, a method for purifying cGAMP comprises: providing a mixture of cGAMP and at least one other compound chosen from dsDNA and cGAS; separating cGAMP from dsDNA and cGAS by ultrafiltration; purifying cGAMP using ion exchange chromatography; and removing salt from cGAMP by lyophilization.

In some aspects, a method for enzymatically synthesizing and purifying cGAMP comprises: providing recombinant cGAS; combining cGAS with ATP, GTP, and dsDNA to synthesize cGAMP; separating cGAMP from dsDNA and cGAS by ultrafiltration; purifying cGAMP using ion exchange chromatography; and removing salt from cGAMP by lyophilization.

The method described above can also be used to synthesize a new derivative of cGAMP called cGAsMP from ATP phosphorothioate and GTP using recombinant cGAS. cGAsMP is not a natural product.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIG. 1 provides the cGAMP/STING pathway in innate immunity against cytosolic dsDNA.

FIGS. 2A-B show the synthesis of cGAMP using recombinant cGAS FIG. 2A shows analysis of enzymatically-synthesized cGAMP by ion exchange chromatography before purification. FIG. 2B illustrates the analysis of purified cGAMP by ion exchange chromatography.

FIGS. 3A-C show that cGAMP induces the expression of IFN-β in cells and in mice. FIG. 3A is an IFN-β reporter assays showing that CDNs differentially regulated the induction of IFN-β in THP1 cells. FIG. 3B is an IFN-β ELISA of THP1 cells treated with cGAMP (black) and 3′,5′ cGAMP (gray). FIG. 3C is an IFN-β ELISA of sera from mice injected with cGAMP.

FIG. 4 shows multiplex cytokine assays, showing that cGAMP induces the expression of a wide spectrum of cytokines and chemokines in THP1 cells.

FIG. 5 provides microarray analysis of gene expression in THP1 cells stimulated by cGAMP. The expression level is indicated by log₂ of the relative expression level, from −7 to 7 colored green to red.

FIG. 6 shows that cGAMP exhibits antitumor activity against several human tumor cell lines. FIG. 6A is an MTT assays showing that cGAMP suppresses the growth of neuronal cancer cell line SF539. FIG. 6B is an MTT assays showing that cGAMP suppresses the growth of renal cancer cell line A498. Controls (white) are cancer cell lines from the same type of tissues.

FIGS. 7A-B show that cGAMP induces the expression of IFN-β in two cGAMP responsive cancer cell lines. FIG. 7A shows that cGAMP induces IFN-β in renal cancer cell line A498. FIG. 7B shows that cGAMP induces IFN-β in CNS cancer cell line SF539.

FIG. 8 shows that the leukemia cell line SR responds to cGAMP treatment but not to IFN-β treatment. (A). MTT assays of leukemia cell lines SR and CCRF-CEM treated with cGAMP. (B). MTT assays of the two leukemia cell lines treated with IFN-β.

FIGS. 9A-M provide a comparison of STING expression levels in normal patients compared to cancer samples. The figures show that STING is expressed at higher levels in cancer patients. Each figure was prepared with a different data set.

FIGS. 10A-B shows cGAS (also known as MB21D) expression magnitude in five subtypes of breast cancer. FIGS. 10C-F plot survival probability against relapse-free survival (in years) for patients with lower and higher amounts of cGAS expression.

FIGS. 11A-B provide data demonstrating that production of cGAMP is too low in certain cancer patients. FIG. 11A shows staining of breast cancer and normal breast tissue with an anti-cGAS antibody. FIG. 11B also quantitates reduced cGAS expression in breast cancer as compared to normal breast tissue.

FIGS. 12A-B provide structural drawings, with FIG. 12A providing the chemical structure of 2′5′-cGAMP and FIG. 12B providing the chemical structure of 2′5′-cGAsMP, a non-naturally occurring derivative of cGAMP.

FIGS. 13A-B show that both cGAMP and cGAsMP can induce IFN-β beta production, but that cGAsMP, a derivative of cGAMP, has enhanced potency. FIG. 13A shows IFN-β ELISA results of THP1 cells treated with cGAMP and cGAsMP. FIG. 13B shows results of IFN-β reporter assays of THP1 cells treated with cGAMP and cGAsMP. cGAsMP is a new compound not occurring in nature.

FIG. 14A shows the results in an MTT of treatment of a neuronal cancer cell line SF539 treated with cGAMP and cGAsMP. FIG. 14B shows the results in an MTT assay of a leukemia cell line SR treated with cGAMP and cGAsMP.

FIGS. 15A-D show the results of several in vivo mouse cancer model experiments evaluating the ability of cGAMP to reduce tumor growth as compared to vehicle alone in seeded colon cancer, seeded breast cancer, and spontaneous breast cancer mouse models.

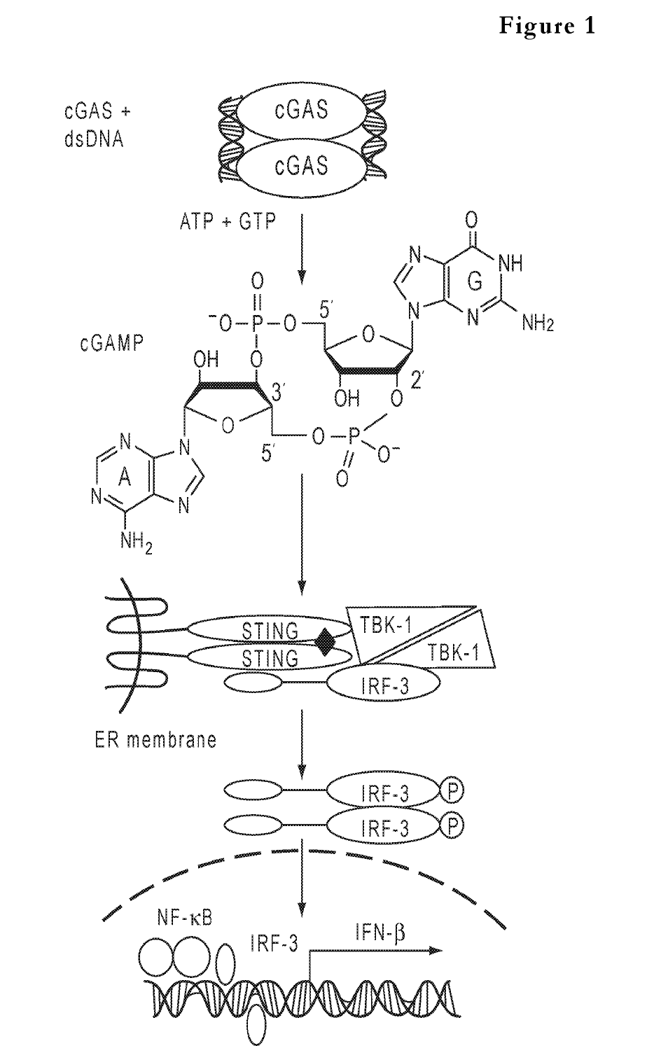

### DESCRIPTION OF THE EMBODIMENTS

I. Enzymatic Synthesis and Purification of cGAMP and cGAsMP

cGAMP and cGAsMP may be enzymatically synthesized using cGAS (encoded by the MB21D1 gene). cGAS may be mixed with ATP (for the synthesis of cGAMP) or ATP phosphorothioate (for the synthesis of cGAsMP), and GTP substrates, optionally in the presence of an ingredient to reduce nonspecific interactions (such as salmon sperm DNA) and buffers, salts, and antioxidants (such as MgCl₂, HEPES buffer, NaCl, and β-mercaptoethanol).

This synthesis method offers improvements from the prior art as, in some instances, it does not require modified nucleotides. It also may be conducted in single step and in a single pot (whether the synthesis alone or the synthesis portion of the combined synthesis and purification method).

The precipitants in the sample may be removed by centrifugation. cGAMP may be separated from the enzyme and dsDNA by ultrafiltration (such as with a Amicon centrifugal filter with a 10 kD cutoff). cGAMP may be further purified using ion exchange chromatography using a Q Sepharose column and eluted from the column with an ammonium acetate solution. Alternatively, cGAMP or cGAsMP can be purified by gel filtration chromatography using a Superdex peptide column eluted with pure water or an ammonium acetate solution. If cGAsMP is being prepared, purification of the active stereoisomer of cGAsMP may be achieved through one additional purification step, namely a gel filtration chromatography step using a Superdex peptide column eluted with an ammonium acetate solution (such as 0.05 M). cGAsMP can be used as a racemic mixture or the active stereoisomer can be used alone.

In some instances, the enzymatic synthesis method provides high yields and a high purity product so that the product can easily be purified by ultrafiltration followed by ion exchange chromatography.

In some embodiments, this purification scheme can purify cGAMP from dsDNA, cGAS, ATP, GTP and/or other byproducts. Additionally, in some embodiments, up to 1 gram quantities of cGAMP may be synthesized and purified through this route. In some embodiments, kilogram level quantities may be prepared, for example 10 kilograms. Because the synthesis may be conducted in a single step and in a single pot and the purified through scalable techniques such as ultrafiltration and column chromatography, the size of the columns etc. may be scaled to the quantities of cGAMP desired for production. These improvements may improve the yield, convenience, and lower the cost of the production and/or purification of cGAMP.

### II. Methods of Treatment of Cancer

A. Types of Cancer

In one embodiment, the methods include a method of treating cancer by administering cGAMP or cGAsMP to a patient having cancer and allowing the cGAMP or cGAsMP to treat the cancer. In one embodiment, the cancer has an increased STING expression level. In another embodiment, the cancer has a decreased cGAS expression level. In another embodiment, the cancer has both an increased STING expression level and a decreased cGAS expression level.

The increased STING expression level may be at least about 1, 1.2, 1.25, 1.3, 1.4, 1.5, 1.6, 1.7, 1.8, 1.9, 2.0, 2.25, 2.5, 2.75, 3.0, 3.25, 3.5, 3.75, 4.0, 4.25, or 4.5 fold higher than an average level in normal cells. STING expression levels in a cancer specimen may be compared to normal levels in a normal patient pool using immunohistochemical staining by employing an antibody specific for STING that may be conjugated to a moiety that enables its visualization (such as an enzyme, including alkaline phosphatase or horseradish peroxidase, or a flurophore, such as fluorescein or rhodamine). The normal patient pool data may be stored in a database and may be used to compare cancer specimens at a different time point.

cGAS/MB21D1 catalyzes ATP and GFP to produce cGAMP, which serves as a ligand for STING. Given that STING is overexpressed in cancer, and while not being bound by theory, cGAS may not be expressed normally in certain cancers or may not function normally. In some cancers, cGAS levels were reduced as compared to either normal patients or as compared to other cancer samples. Lower cGAS levels are associated with poorer outcomes and higher cGAS levels are associated with more positive outcomes. Thus, restoring the level of the cGAS pathway in tumors may help to restrain tumor cell growth through STING-dependent pathways.

The decreased cGAS expression level may be within the lower about 75%, 70%, 65%, 60%, 55%, 50%, 45%, 40%, 35%, 30%, 25%, 20%, 15%, or 10% of patients, when evaluating the cGAS level in a pool of patients having cancer or in a pool of subjects including both cancer patients and normal patients. The cGAS level of which 75% patients have lower expression will be set as a standard given that this low cGAS expression population has reduced survival.

Increased STING expression has been demonstrated in at least the following cancer types: leukemia (including, but not limited to, acute myeloid leukemia, chronic myelogenous leukemia, and pro-B acute lymphoblastic leukemia), lymphoma (including, but not limited to, activated B-cell-like diffuse large B-cell lymphoma, diffuse large B-cell lymphoma, follicular lymphoma, anaplastic large cell lymphoma, angioimmunoblastic T-cell lymphoma, ALK-positive, Burkitt's lymphoma, Hodgkin's lymphoma, nodular lymphocyte predominant Hodgkin's lymphoma, T-cell/histiocyte-rich large B-cell lymphoma, and germinal center B-cell-like diffuse large B-cell lymphoma), gastric cancer (diffuse gastric adenocarcinoma, gastric intestinal type adenocarcinoma, and gastric mixed adenocarcinoma), esophageal cancer (Barrett's esophagus, esophageal squamous cell carcinoma, and esophageal adenocarcinoma), colorectal cancer, pancreatic cancer, embryonal carcinoma, mixed germ cell tumor, seminoma, teratoma, yolk sac tumor, testicular teratoma, thyroid cancer, renal carcinoma, melanoma, glioblastoma, tongue carcinoma, breast cancer, oral cavity carcinoma, oropharyngeal carcinoma, tonsillar carcinoma.
B. Dosage and Routes of Administration

cGAMP or cGAsMP may be administered to patients in need thereof through a number of routes of administration. In one embodiment, the cGAMP or cGAsMP may be administered through a parenteral route of administration, including but not limited to intravenous, intraarterial, intramuscular, intracerebral, intracerebroventicular, intrathecal, and subcutaneous. In another embodiment, the cGAMP or cGAsMP may be provided by inhalation, topically, or orally.

cGAMP or cGAsMP may be prepared into a pharmaceutical preparation. In one embodiment, sterile saline may be used in order to prepare a pharmaceutically acceptable preparation. The cGAMP or cGAsMP may also be prepared in lyophilized form and dissolved in sterile saline for injection before administration to a patient.

A dosage of from about 0.1 to about 1 mg/kg of body weight may be used for the treatment of patients. In some embodiments, the dosage may be about 0.1 mg/kg, 0.5 mg/kg, or 1.0 mg/kg.

### EXAMPLES

### Example 1

### The Enzymatic Synthesis and Purification of cGAMP

A. Expression and Purification of Recombinant cGAS

The cDNA clones of human and mouse cGAS (referred to as hcGAS and mcGAS, respectively) were purchased from Open Biosystems Inc. Full-length and catalytic domains of hcGAS and mcGAS were subcloned into a modified pET-28(a) (Novagen) vector with an N-terminal 6× His followed by a SUMO tag. The recombinant His₆-SUMO-hcGAS (157-522) and His₆-SUMO-mcGAS (142-507) were expressed in E. coli BL21(DE3) induced with 1 mM of isopropyl β-D-1-thiogalactopyranoside (IPTG) at 15° C. overnight.

The cells were harvested by centrifugation and resuspended in a lysis buffer containing 50 mM Tris, 300 mM NaCl at pH 8.0. The cell lysate was centrifuged at 4000 rpm for 10 min and the supernatant was collected. The samples were centrifuged again at 16,000 rpm for 30 min. The supernatant was then loaded on a Ni-NTA column and washed with a buffer containing 500 mM NaCl, 20 mM Tris, 25 mM imidazole at pH 7.5. The protein was eluted with a buffer containing ˜250 mM imidazole, 150 mM NaCl, 20 mM Tris-HCl at pH 7.6. Fractions containing cGAS were pooled and 5 mM DTT were added to the sample. The SUMO tag was cleaved with sumo protease overnight. The samples were analyzed by SDS-PAGE to confirm that the cleavage was complete. The cleaved cGAS sample was concentrated and purified again using a Superdex200 (16×60) column (GE Healthcare) eluted with a buffer containing 20 mM Tris-HCl, 500 mM NaCl at pH 7.5 for human cGAS and a buffer containing 20 mM Tris-HCl, 150 mM NaCl at pH 7.5 for mouse cGAS. Fractions from the gel filtration column were analyzed by SDS-PAGE and fractions containing cGAS were pooled and 5 mM β-mercaptoethanol was added to the samples. Purified cGAS was concentrated to ˜15 mg/ml, aliquoted, frozen in liquid nitrogen, and stored in −80° C. The yield of the recombinant enzyme is around 4 mg per liter of bacterial culture. These enzymes were used for biosynthesis of cGAMP.
B. Enzymatic Synthesis and Purification of cGAMP

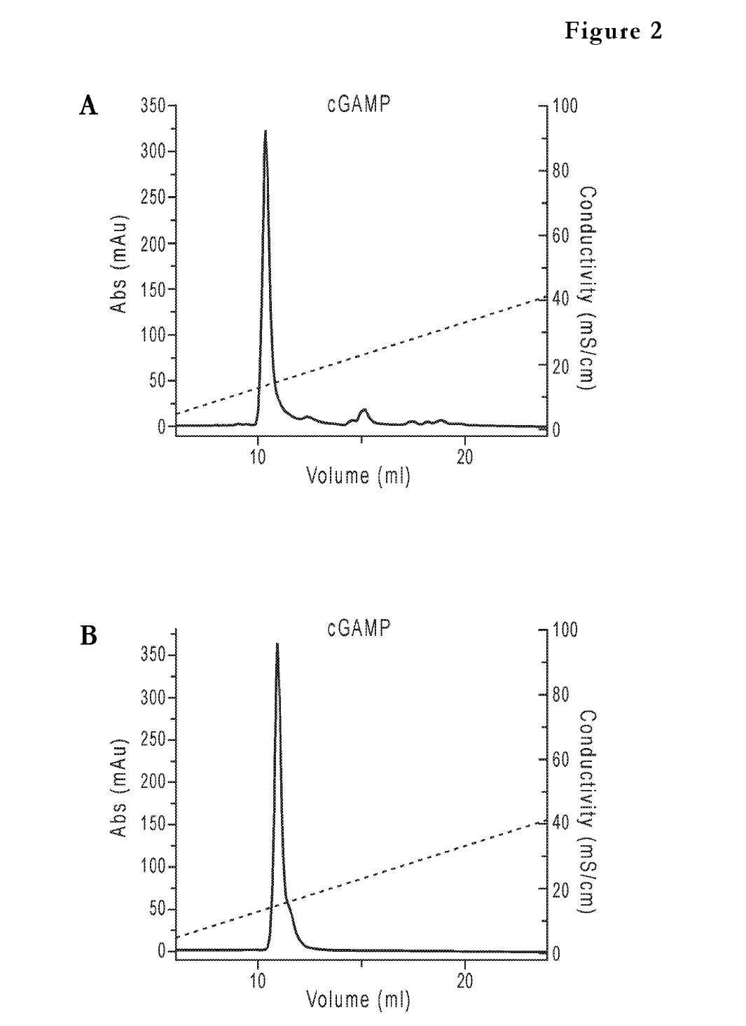

The reaction mixture for the biosynthesis of cGAMP contains 10 μM recombinant cGAS, 0.2 mg/ml of salmon sperm DNA, 5 mM ATP, 5 mM GTP, 5 mM MgCl₂, 20 mM HEPES buffer of pH 7.5, 150 mM NaCl, and 10 mM β-mercaptoethanol. The mixture was incubated for 12 hours at 37° C. until the substrates of ATP and GTP were exhausted. The sample was analyzed by ion exchange chromatography using a MonoQ column (GE Healthcare) to confirm the formation of cGAMP. The sample was then clarified by centrifugation at 4000×g for 15 minutes to remove insoluble precipitant formed during the reaction. The enzyme and dsDNA were separated from the reaction product by ultrafiltration using centrifugal filter with a 10 kD pore size (Millipore). cGAMP was further purified by ion exchange chromatography using a Q Sepharose column (FIG. 2). After washing with a 0.1 M ammonium acetate solution, cGAMP was eluted from the column with a solution containing 0.3 M ammonium acetate. The eluted cGAMP was lyophilized and stored at −80° C. Under optimal reaction conditions, more than 80% ATP and GTP are converted into cGAMP. The yield of cGAMP is ˜5 mg for each milligram of recombinant cGAS used. This protocol has been used routinely to synthesize cGAMP at 50-100 mg scale in the lab and can be scaled up to larger scale for different needs.

### Example 2

### cGAMP Stimulates the Expression of IFN-β and Other Cytokines

A. cGAMP Induces the Expression of IFN-β in Cells and in Mice

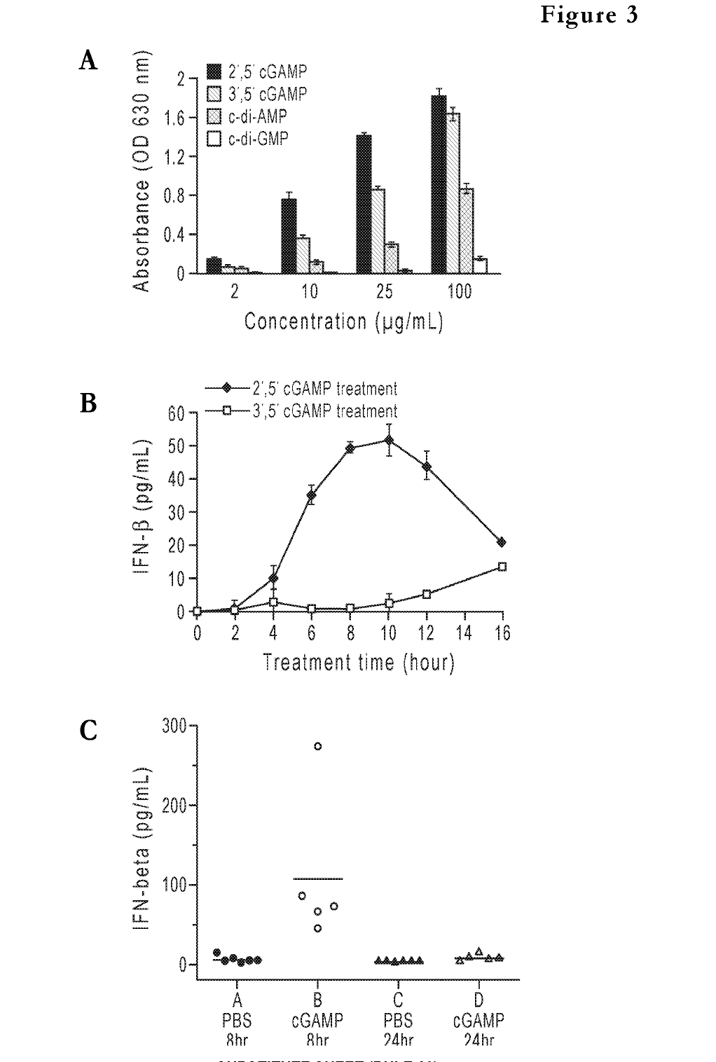

To confirm that cGAMP can induce the expression of IFN-β, we stimulated human monocytes THP1 blue cells with cGAMP and other three cyclic dinucleotides added to the culture media. We observed that cGAMP is very potent in inducing the expression of IFN-β reporter (FIG. 3A). In contrast, 3′,5′ cGAMP has lower activity (FIG. 3A). Cyclic di-AMP and c-di-GMP exhibit even lower activities (FIG. 3A). To confirm these results, we analyzed IFN-β levels in the culture supernatant by ELISA. We observed rapid responses to cGAMP by the THP1 cells. The induction of IFN-β peaked at 8-10 hours post stimulation (FIG. 3B). In contrast, the response to 3′,5′ cGAMP is much weaker (FIG. 3B). Furthermore, we analyzed the induction of IFN-β by cGAMP in mice. We observed rapid responses in mice after intravenous (i.v.) injection of cGAMP (FIG. 3C) at a dosage of 100 μg/mice.
B. cGAMP Upregulates a Wide Spectrum of Cytokines and Chemokines

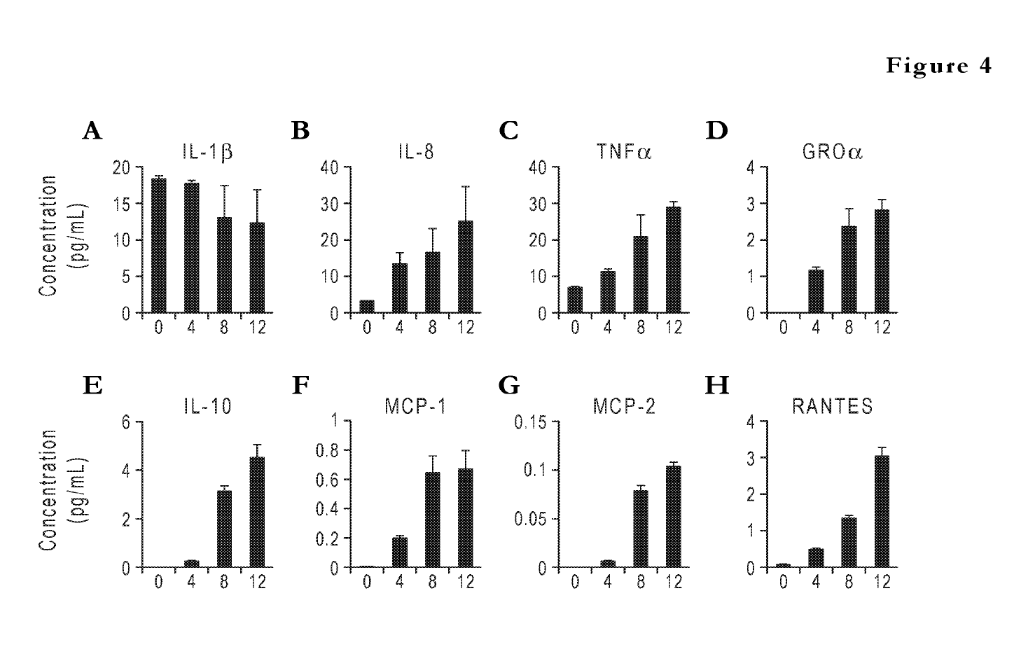

As a novel second messenger in innate immunity, it was only known that cGAMP stimulates the expression of type I interferons. Our NF-κB reporter assays shows that cGAMP or the over expression of cGAS also stimulate the activation of NF-κB. It is likely the stimulation of STING by cGAMP also regulates the induction of other cytokines or chemokines. Indeed, we have observed the up-regulation of IL-8, TNF-α, GROα, IP-10, MCP-1, MCP-2, and RANTES by cGAMP in THP1 cell by multiplex cytokine assays (FIG. 4). However, cGAMP does not up-regulate the expression of IL-1β, a major inflammatory cytokine.

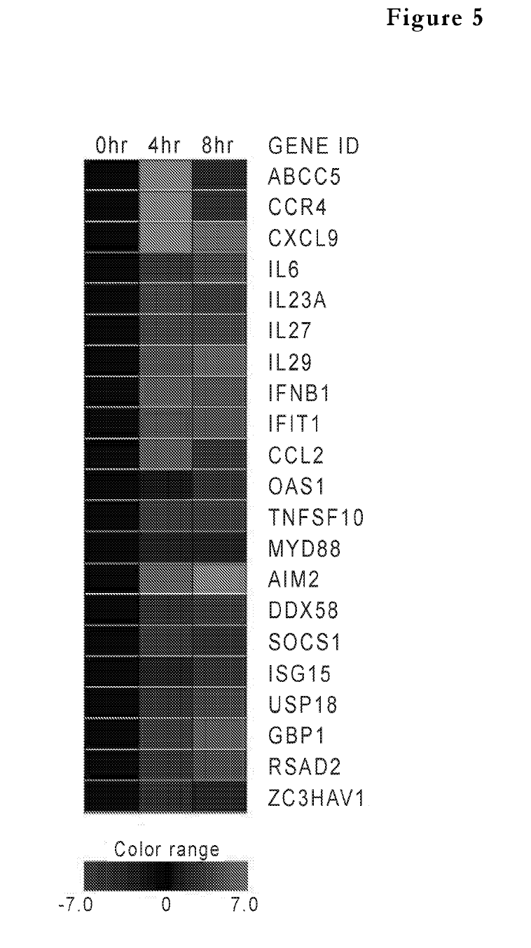

To investigate the effect of cGAMP on genome-wide gene expression, we have performed microarray analysis of THP1 cells treated with 20 μg/ml of cGAMP at 4 hours and 8 hours post treatment. These microarray data revealed that cGAMP up-regulates over 200 genes, many of which are interferon inducible genes and various cytokine genes (FIG. 5).

### Example 3

### The Antitumor Activities of cGAMP

A. The Antitumor Activities of cGAMP

First, we confirmed the binding interaction between cGAMP and human STING by isothermal titration calorimetry (ITC). Ligand binding studies showed that cGAMP binds human STING with an affinity of ˜60 nM, which is ˜50 times higher than its binding affinity for the bacterial cyclic dinucleotide c-di-GMP Next, we conducted the NCI60 antitumor screen using the enzymatically-synthesized cGAMP. Of the sixty human cancer cell lines (NCI60) tested, a single dose of 10 μM cGAMP effectively inhibited the growth of CNS cancer cell line SF539, renal cancer cell line A498, and leukemia cell line SR; however, only one concentration was tested and the concentration selected for initial testing may have been too low. Higher doses are expected to provide beneficial results in a larger number of the tested cell lines.

The cell lines tested were: NSCLC_NCIH23, NSCLC_NCIH522, NSCLC_A549ATCC, NSCLC_EKVX, NSCLC_NCIH226, NSCLC_NCIH332M, NSCLC_H460, NSCLC_HOP62, NSCLC_HOP92, COLON_HT29, COLON_HCC-2998, COLON_HCT116, COLON_SW620, COLON_COLO205, COLON_HCT15, COLON_KM12, BREAST_MCF7, BREAST_MCF7ADRr, BREAST_MDAMB231, BREAST_HS578T, BREAST_MDAMB435, BREAST_MDN, BREAST_BT549, BREAST_T47D, OVAR_OVCAR3, OVAR_OVCAR4, OVAR_OVCAR5, OVAR_OVCAR8, OVAR_IGROV1, OVAR_SKOV3, LEUK_CCRFCEM, LEUK_K562, LEUK_MOLT4, LEUK_HL60, LEUK_RPMI8266, LEUK_SR, RENAL_UO31, RENAL_SN12C, RENAL_A498, RENAL_CAKI1, RENAL_RXF393, RENAL_7860, RENAL_ACHN, RENAL_TK10, MELAN_LOXIMVI, MELAN_MALME3M, MELAN_SKMEL2, MELAN_SKMEL5, MELAN_SKMEL28, MELAN_M14, MELAN_UACC62, MELAN_UACC257, PROSTATE_PC3, PROSTATE_DU145, CNS_SNB19, CNS_SNB75, CNS_U251, CNS_SF268, CNS_SF295, and CNS_SF539.

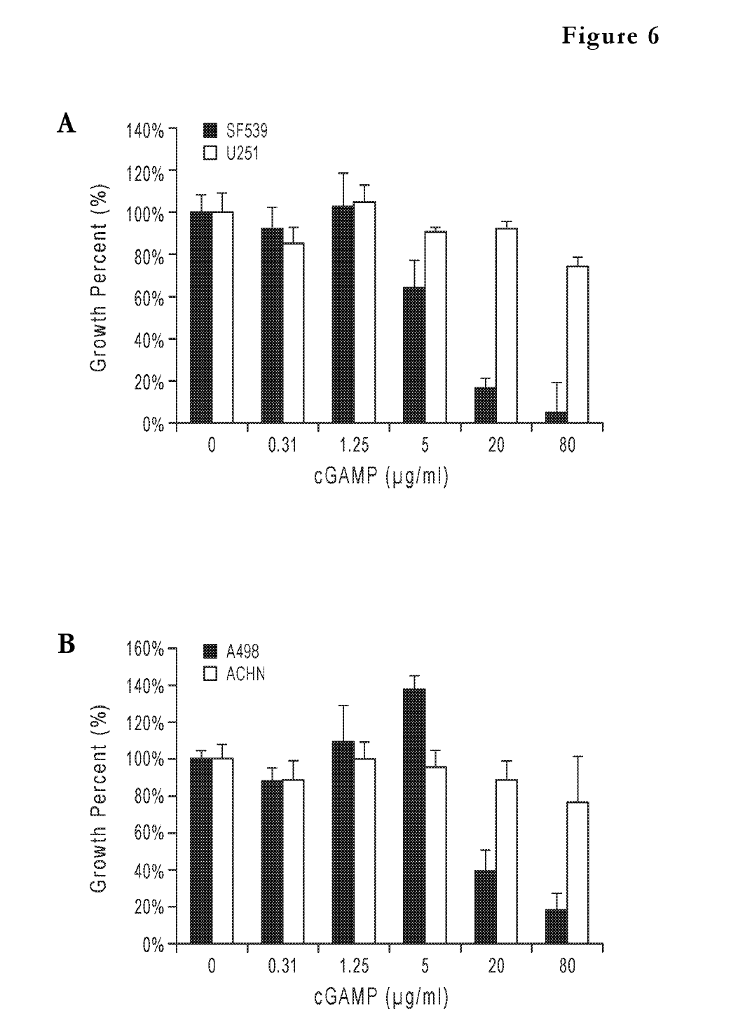

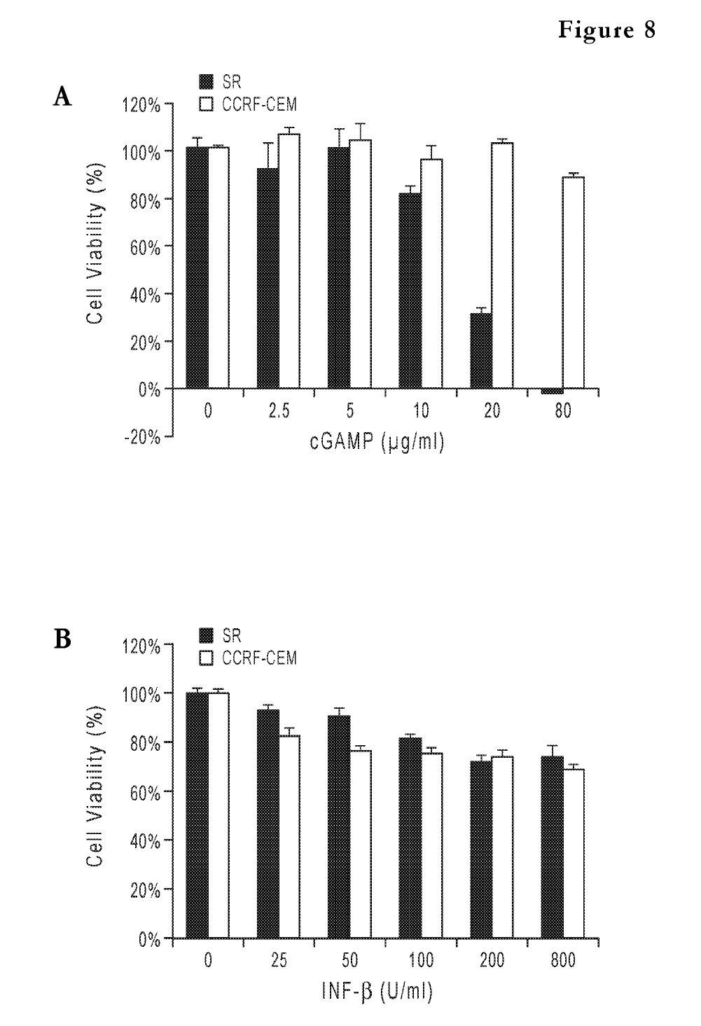

We have reproduced the results from the NCI60 screens and confirmed the antitumor activity of cGAMP in the three cancer cell lines. Three non-responding tumor cell lines from the same type of tissues were used as controls in these studies. After validating the data from the NCI60 screen, we have conducted MTT assays for these three tumor cell lines together with the three control cell lines and observed similar results (FIGS. 6 and 8A). These results clearly demonstrated that cGAMP has direct tumor suppressive activity against certain types of human tumor cells.
B. cGAMP Induces the Expression of IFN-β in Tumor Cells

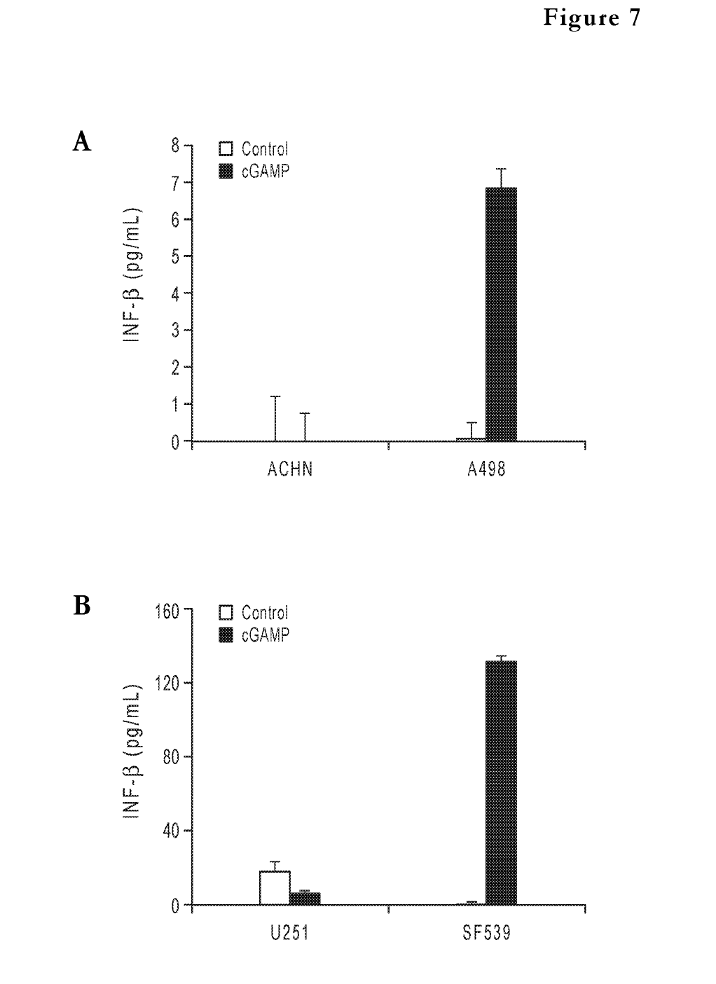

To examine whether STING-mediated signaling plays a role in the antitumor activity of cGAMP, we analyzed the microarray data available for the NCI60 cell lines. We found that the three cell lines that responded to cGAMP express higher levels of STING, while the control cell lines express lower levels of STING. Microarray data from NCI for the 60 cell lines shows higher levels of STING in the cGAMP responding tumor cell lines compared to the non-responding control cell lines we used. This suggests that STING mediated signaling likely plays a key role in the antitumor activity of cGAMP. Consistent with these observations, we have observed the induction of IFN-β by cGAMP in the two responding cell lines (FIG. 7). In contrast, inductions of IFN-β in the two control cell lines tested are quite low (FIG. 7). These data suggestion the cGAMP/STING pathway is likely involved in the antitumor activity of cGAMP.

To test whether IFN-β induced by cGAMP mediates the suppression of tumor growth, we have treated the three tumor cell lines with either cGAMP or IFN-β alone. We observed that IFN-β suppressed the growth of two tumor cell lines and is almost as potent as cGAMP at the concentrations tested. However, the leukemia cell line SR responds strongly to cGAMP treatment (FIG. 8A), but does not respond very well to IFN-β treatment (FIG. 8B). The control leukemia cell line CCRF-CEM did not respond to the treatment by cGAMP or IFN-β as well (FIG. 8B). These data suggest that although IFN-β plays a critical role in tumor suppression by cGAMP, other factors induced by cGAMP also play important roles in tumor suppression in certain types of cancer cells.

### Example 4

### Identification of Cancer Types Demonstrating Increased STING Expression

Without being bound by theory, we believe that cGAMP executes its anti-tumor function through a STING-dependent pathway. To support this notion, we have analyzed certain genome-wide gene expression databases. Analysis was performed using a number of publicly-archived genome-wide gene expression arrays to examine the expression of the STING gene. Comparison was made between human cancer specimens and normal tissues. The analysis was performed using the Oncomine® Research bioinformatics platform, available from Life Technologies, Thermo Fisher Scientific.

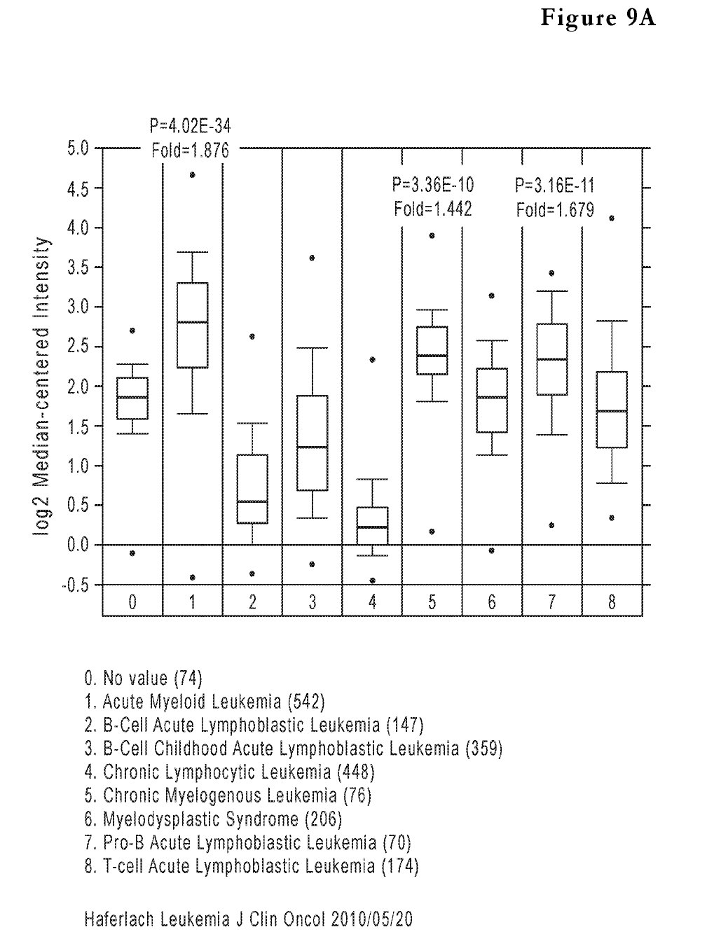

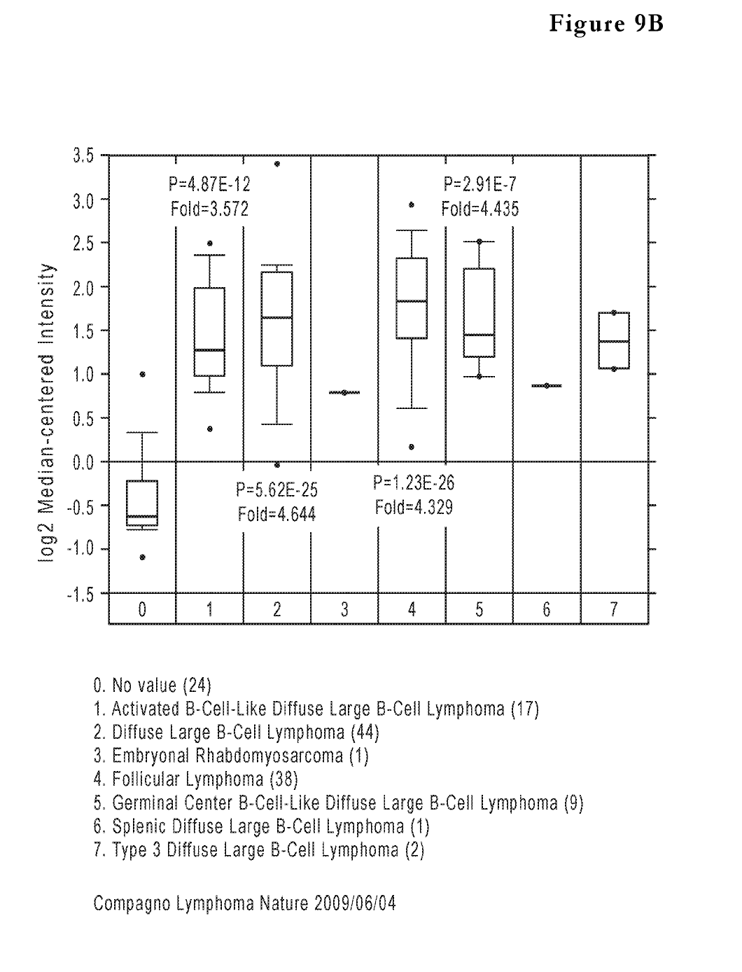

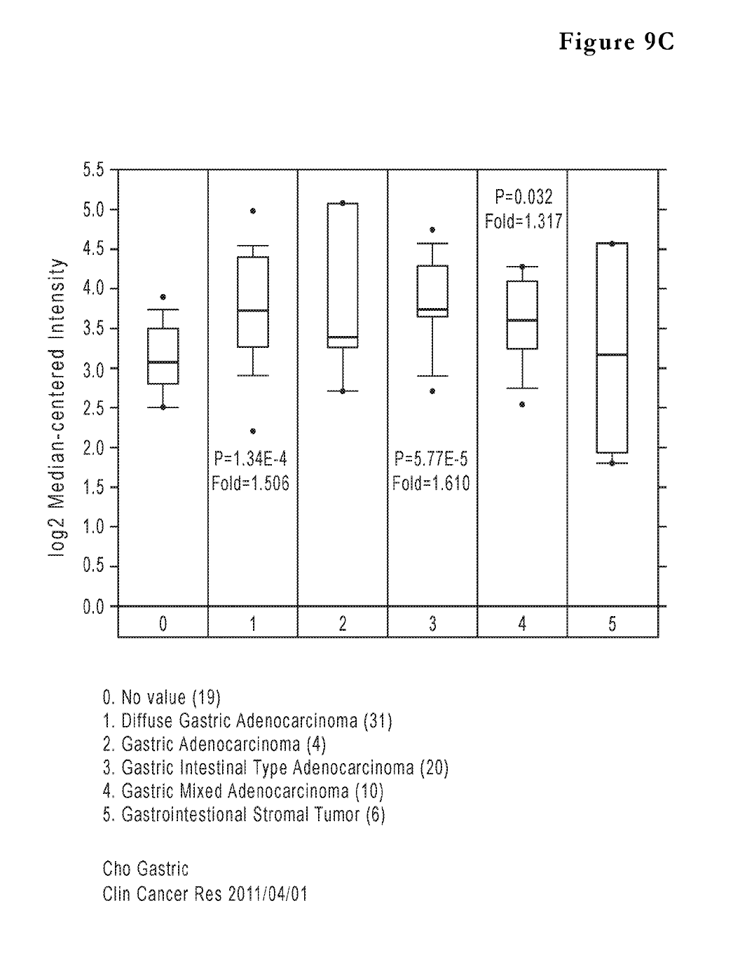

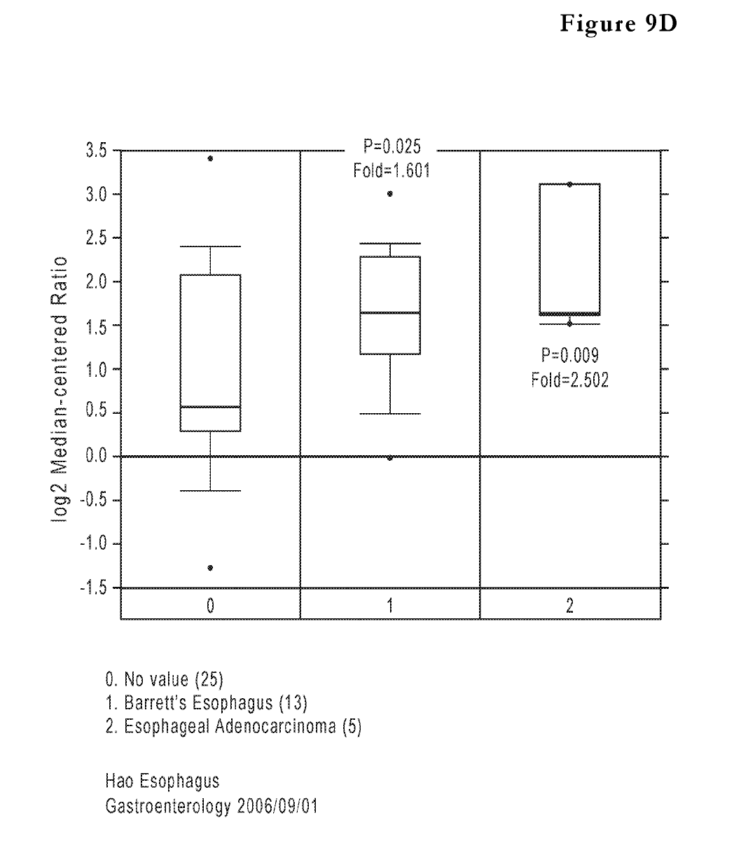

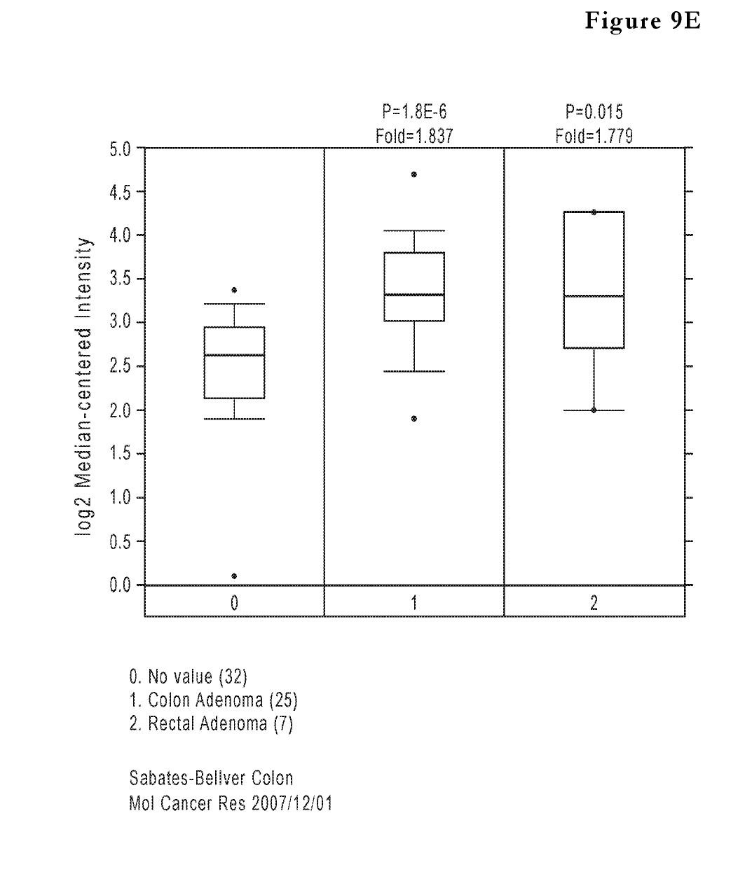

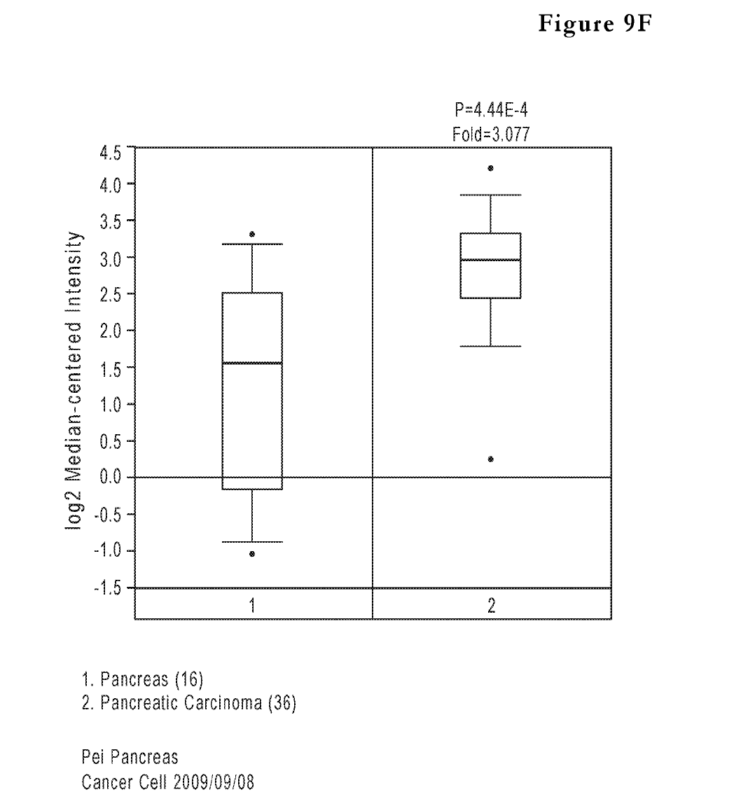

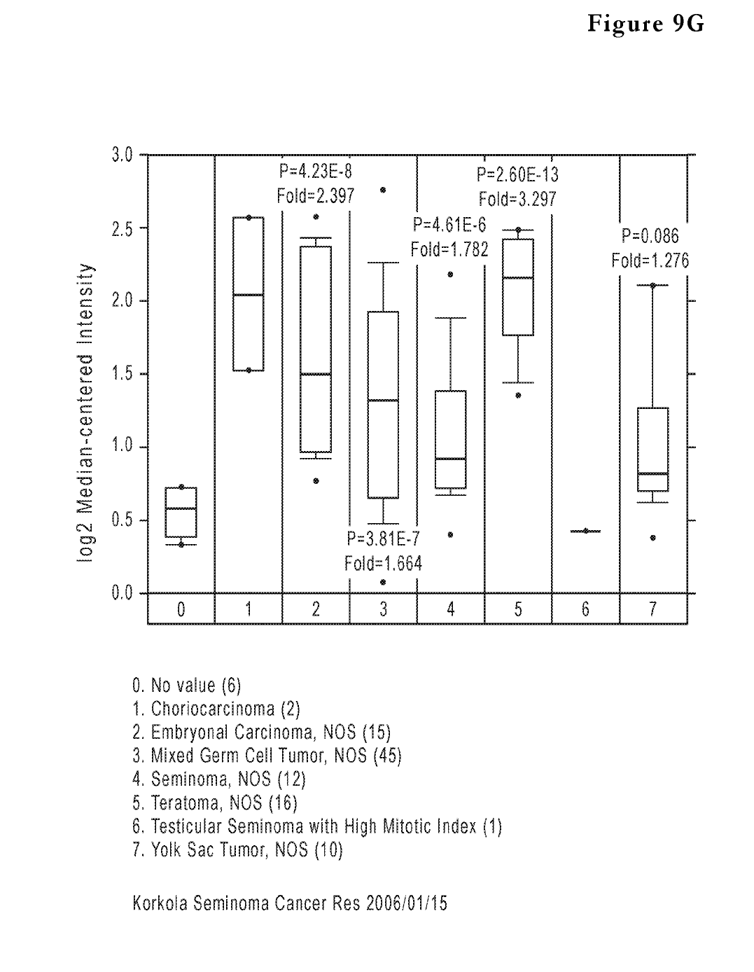

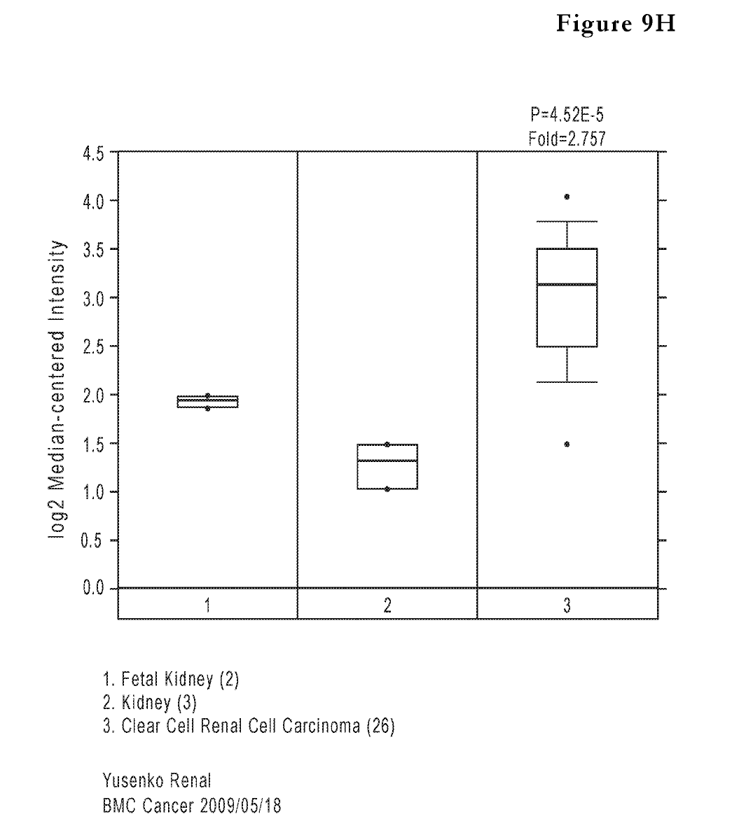

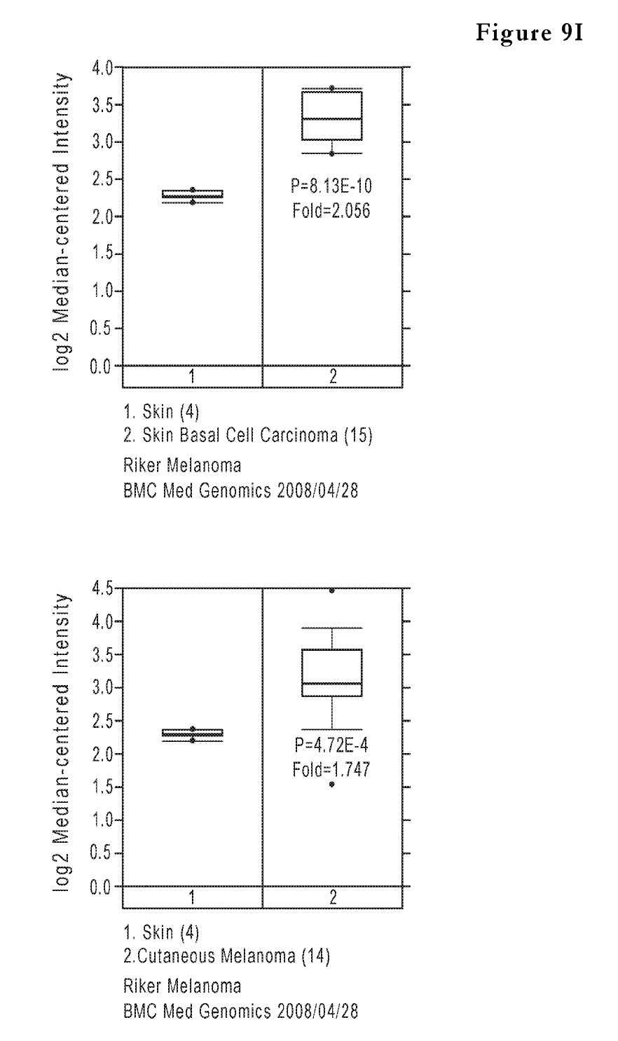

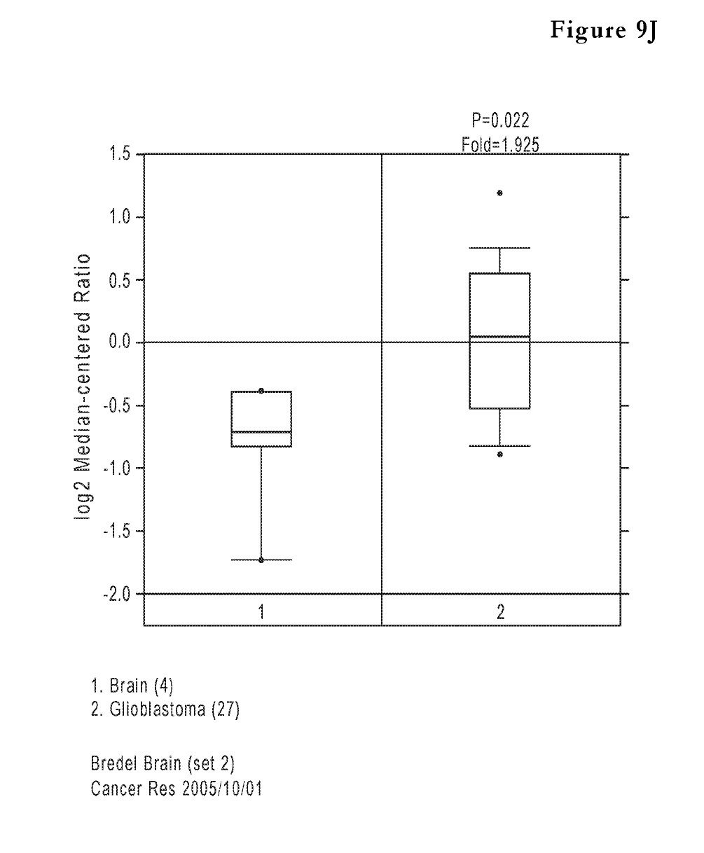

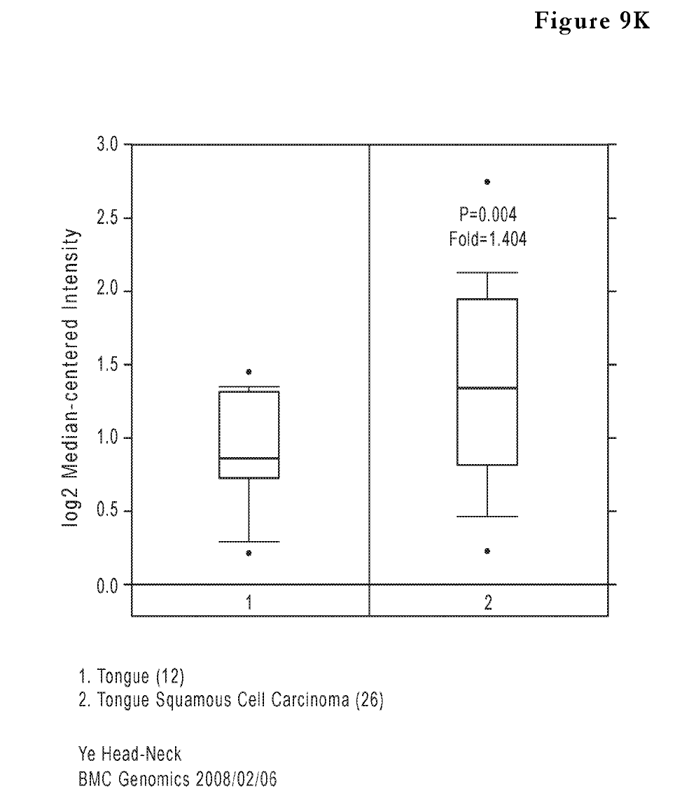

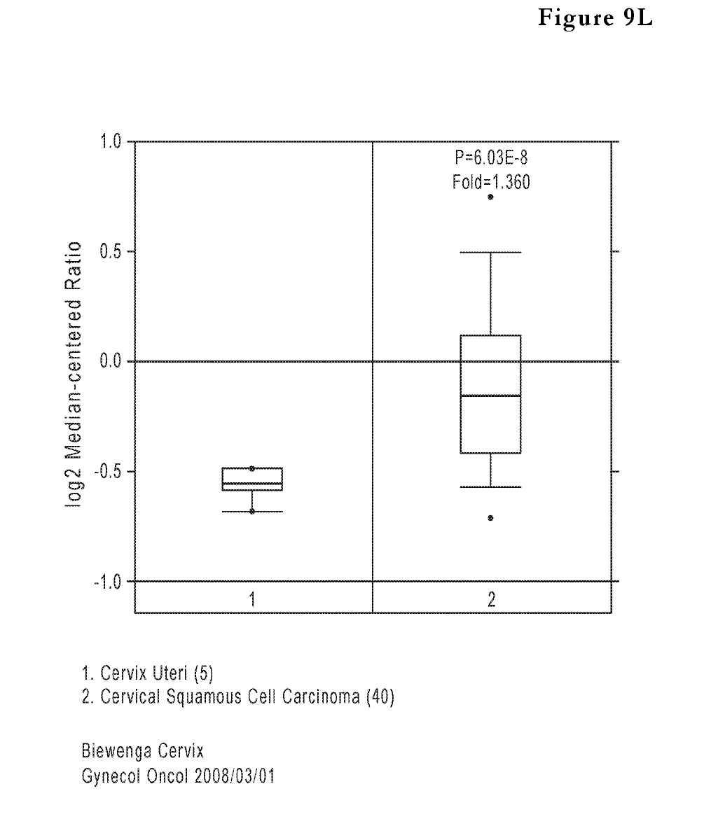

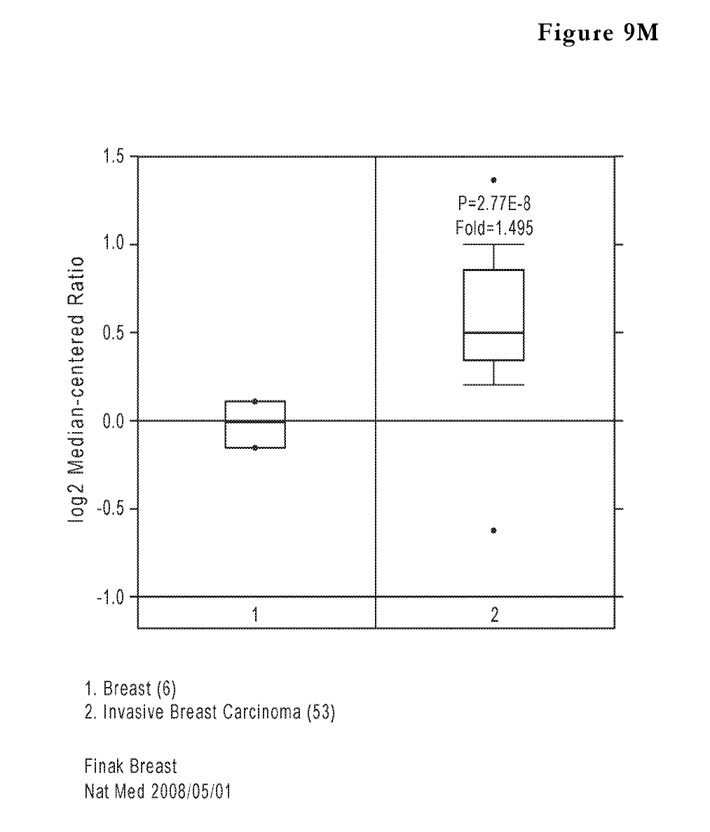

Results of this analysis are presented in FIGS. 9A-M. Increased STING expression was found in the following cancer types: leukemia (including, but not limited to, acute myeloid leukemia, chronic myelogenous leukemia, and pro-B acute lymphoblastic leukemia), lymphoma (including, but not limited to, activated B-cell-like diffuse large B-cell lymphoma, diffuse large B-cell lymphoma, follicular lymphoma, anaplastic large cell lymphoma, angioimmunoblastic T-cell lymphoma, ALK-positive, Burkitt's lymphoma, Hodgkin's lymphoma, nodular lymphocyte predominant Hodgkin's lymphoma, T-cell/histiocyte-rich large B-cell lymphoma, and germinal center B-cell-like diffuse large B-cell lymphoma), gastric cancer (diffuse gastric adenocarcinoma, gastric intestinal type adenocarcinoma, and gastric mixed adenocarcinoma), esophageal cancer (Barrett's esophagus, esophageal squamous cell carcinoma, and esophageal adenocarcinoma), colorectal cancer, pancreatic cancer, embryonal carcinoma, mixed germ cell tumor, seminoma, teratoma, yolk sac tumor, testicular teratoma, thyroid cancer, renal carcinoma, melanoma, glioblastoma, tongue carcinoma, breast cancer, oral cavity carcinoma, oropharyngeal carcinoma, tonsillar carcinoma, and cirrhotic liver.

### Example 5

### Illustration of Reduced cGAS Expression in Cancer

By taking breast cancer as an example, we have shown that the average expression of cGAS gene in tumor is similar to the normal tissue (A). Her2 subtype showed significantly reduced cGAS expression comparing to other subtypes (B). We divided patients into two or three groups based on cGAS expression in their tumor. The decreased cGAS expression level may be within the lower 50%, 45%, 40%, 35%, 30%, 25%, 20%, 15%, or 10% of patients, when evaluating the cGAS level in a pool of patients having cancer or in a pool of subjects including both cancer patients and normal patients. The upper 75% patients with high cGAS expression had improved relapse-free survival and the lower 25% had worst outcome (C). Luminal A and B subtypes are both estrogen-receptor-positive (ER+) and low-grade, with luminal A tumors growing very slowly and luminal B tumors growing more aggressively. The aggressive luminal B subtype is a heterogeneous and complex disease and often develops resistance to existing therapies. High cGAS expression in upper 25% patients in subtype B showed a clear benefit of increased relapse-free survival (D). This result demonstrated that tumors had a heterogeneous expression pattern.

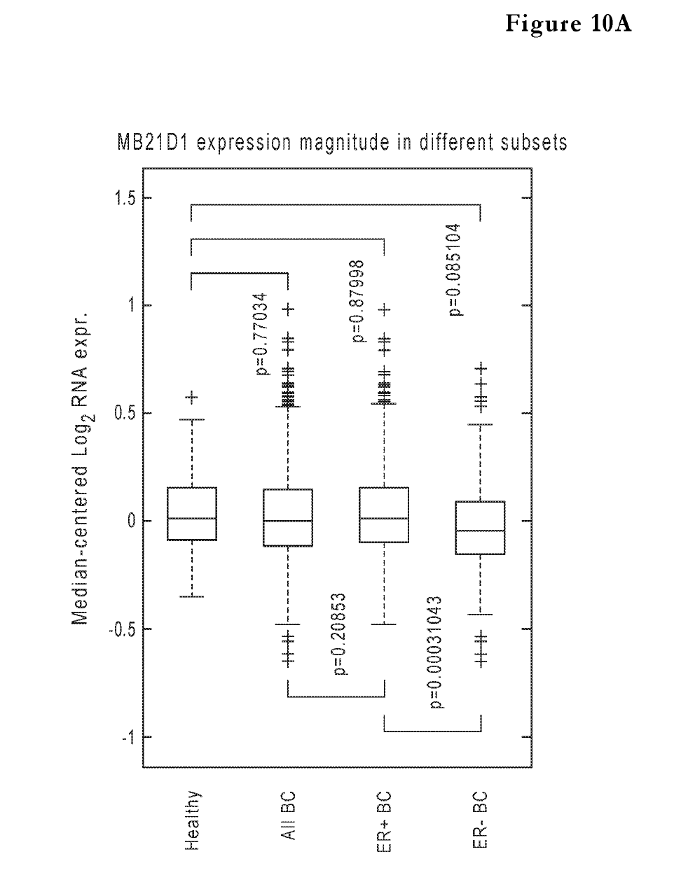

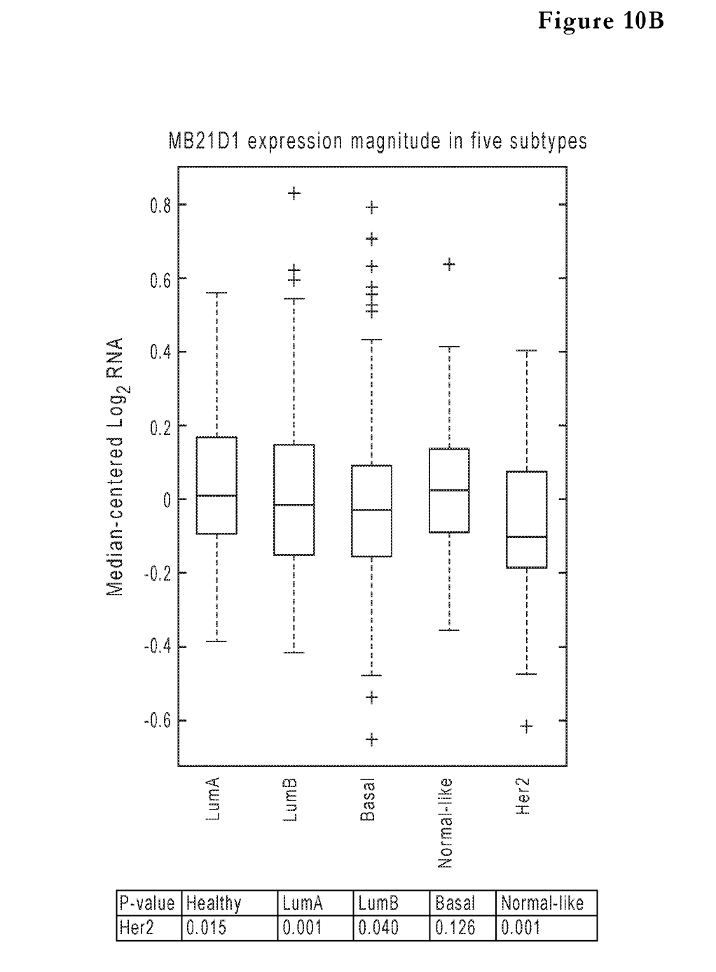

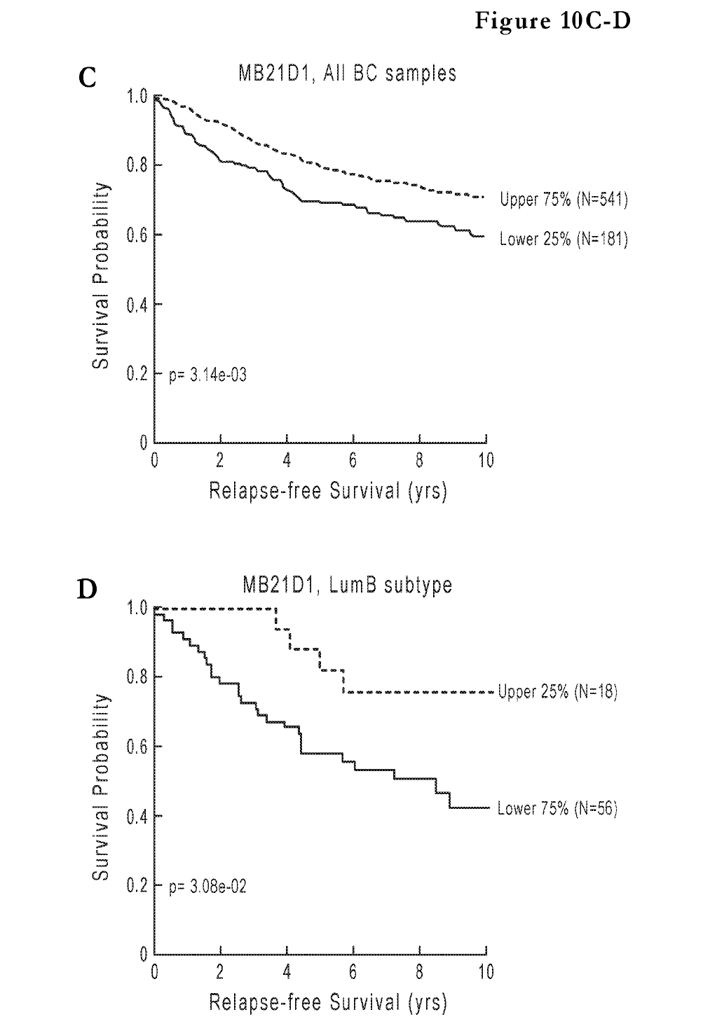

TABLE 1
P-values for FIG. 10B

| P-value | Healthy | LumA | LumB | Basal | Normal-like |
|---|---|---|---|---|---|
| Her2 | 0.015 | 0.001 | 0.040 | 0.126 | 0.001 |

Restoring the level of cGAS in tumors may help to restrain tumor cell growth through STING-dependent pathways. Such, reduced expression of cGAS and/or increased STING expression may facilitate patient selection.

### Example 6

### Staining of Human Breast Specimens

Breast tissue from a normal patient and breast cancer tissue were stained with an anti-cGAS antibody to show the levels of cGAS. Formalin-fixed and paraffin-embedded tumor specimens used in this study were from the tissue bank of LIPOGEN LLC. All tumors were primary and untreated before surgery with complete clinicopathological information. Tumor size was defined as the maximum tumor diameter measured on the tumor specimens at the time of operation. H&E-stained sections of specimens were reviewed and the diagnosis confirmed by an expert gynecologic pathologist. All of the specimens were anonymous and tissues were collected in compliance with institutional review board regulations. Adjacent normal tissues were included for some cancer tissues.

IHC staining for SREBP1 was performed on the paraffin-embedded tissue blocks. Hematoxylin and eosin (H&E) stainings were reviewed to ensure the cancer tissue and normal epitheliums. IHC staining for cGAS was performed on 5 μm thick sections. Briefly, tissue slides were deparaffinized with xylene and rehydrated through a graded alcohol series. The endogenous peroxidase activity was blocked by incubation in a 3% hydrogen peroxide solution for 15 min. Antigen retrieval was performed by immersing the slides in 10 mM sodium citrate buffer (pH 6.0) and maintained at a sub-boiling temperature for 5 min. The slides were rinsed in phosphate-buffered saline and incubated with 10% normal serum to block non-specific staining. The slides were then incubated with the primary antibody (anti-cGAS, from Sigma, Catalog #HPA031700) overnight at 4° C. in a humidified chamber.

All staining was assessed by pathologists blinded to the origination of the samples using a semi-quantitative method. Each specimen was assigned a score according to the intensity of the nucleic and cytoplasmic staining Tissue was scored (H-score) based on the total percentage of positive cells and the intensity of the staining (1+, 2+ or 3+), where H=(% “1+”×1)+(% “2+”×2)+(% “3+”×3). A minimum of 100 cells was evaluated in calculating the H-score.

Statistical analysis. Means of continuous variables for cGAS staining intensity between breast cancer and adjacent normal tissue were compared by one-way analysis of variance (multiple comparisons). The comparison between the clinicopathologic characteristics of breast cancer and cGAS staining intensity was evaluated with the Mann-Whitney U test. All statistical tests were two-sided, and p-values less than 0 05 were considered as statistically significant. The statistical analyses were performed using SPSS 13.0 software (SPSS Inc.).

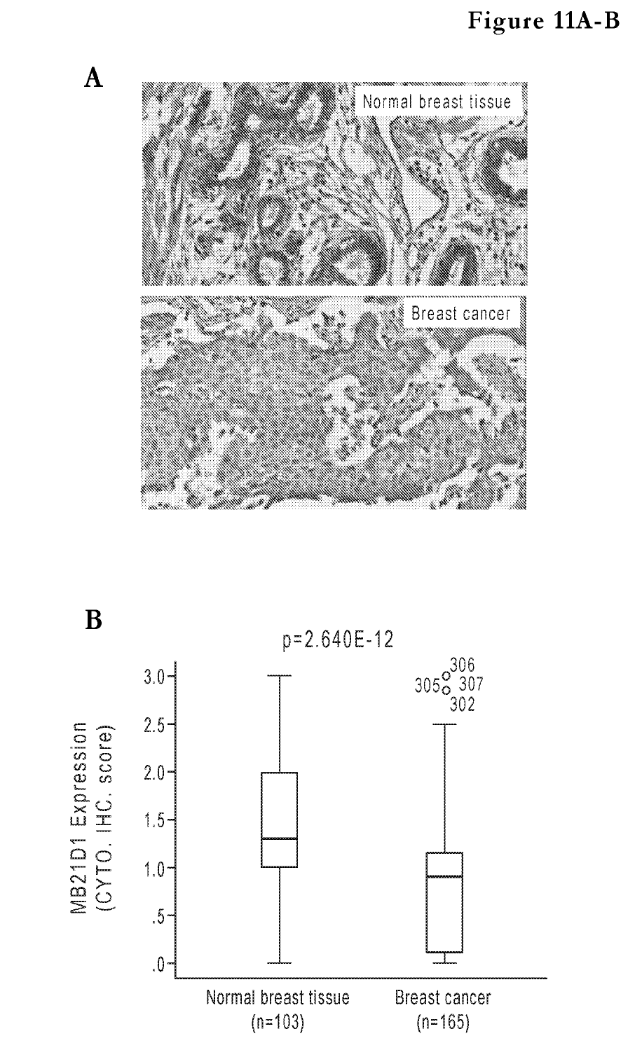

Because cGAS is involved in producing cGAMP, lower levels of cGAS result in lower levels of cGAMP. The breast cancer tissue sample shows reduced staining with the anti-cGAS antibody. See FIG. 11A.

cCAS expression was quantified and the results are provided in FIG. 11B, showing reduced cGAS expression in breast cancer as compared to normal breast tissue.

### Example 7

### The Synthesis and Purification of cGAsMP

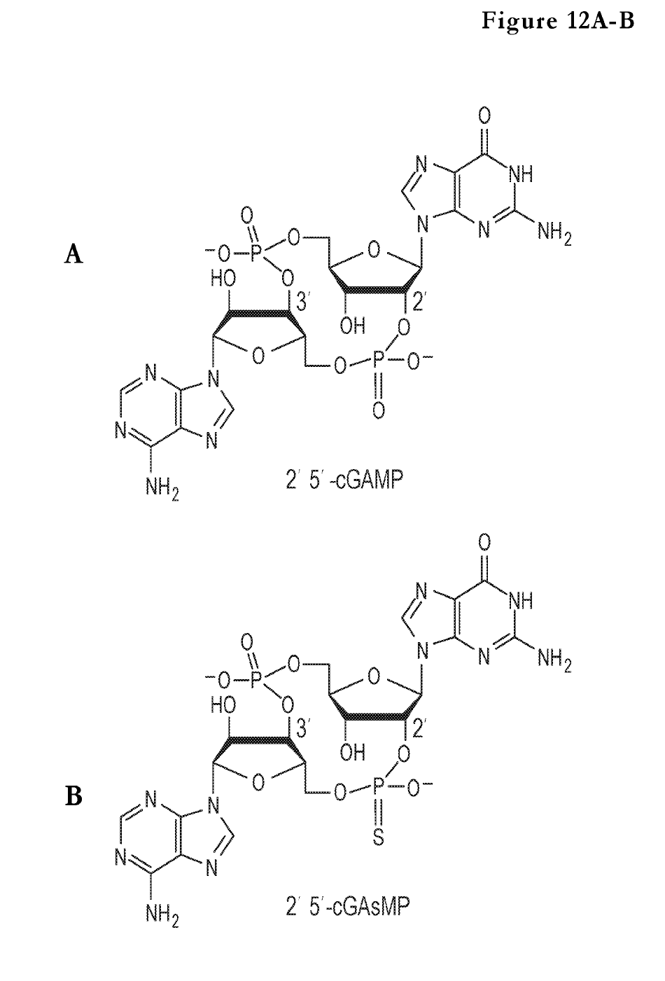

A derivative of 2′5′-cGAMP, 2′5′-cGAsMP, was prepared and the chemical structure for the two compounds are provided in FIGS. 12A-B. cGAsMP can be synthesized using a similar protocol as described for cGAMP in Example 1 from ATP phosphorothioate and GTP. The concentration of the substrates (ATP phosphorothioate and GTP) were 1 mM for cGAsMP synthesis, modified from the protocol for synthesizing cGAMP to improve the yield of cGAsMP; however, the cGAS concentration was unchanged compared to the prior protocol. Purification of the active stereoisomer of cGAsMP is achieved through one additional purification step, namely gel filtration chromatography step using a Superdex peptide column eluted with an ammonium acetate solution (0.05 M). Gel filtration chromatography shows that the purified cGAsMP stereoisomer binds STING, while the other stereoisomer of cGAsMP does not bind STING. Thus, cGAsMP can be used as a racemic mixture or the active stereoisomer can be used alone.

### Example 8

### cGAsMP is More Potent than cGAMP in Inducing IFN-β Expression

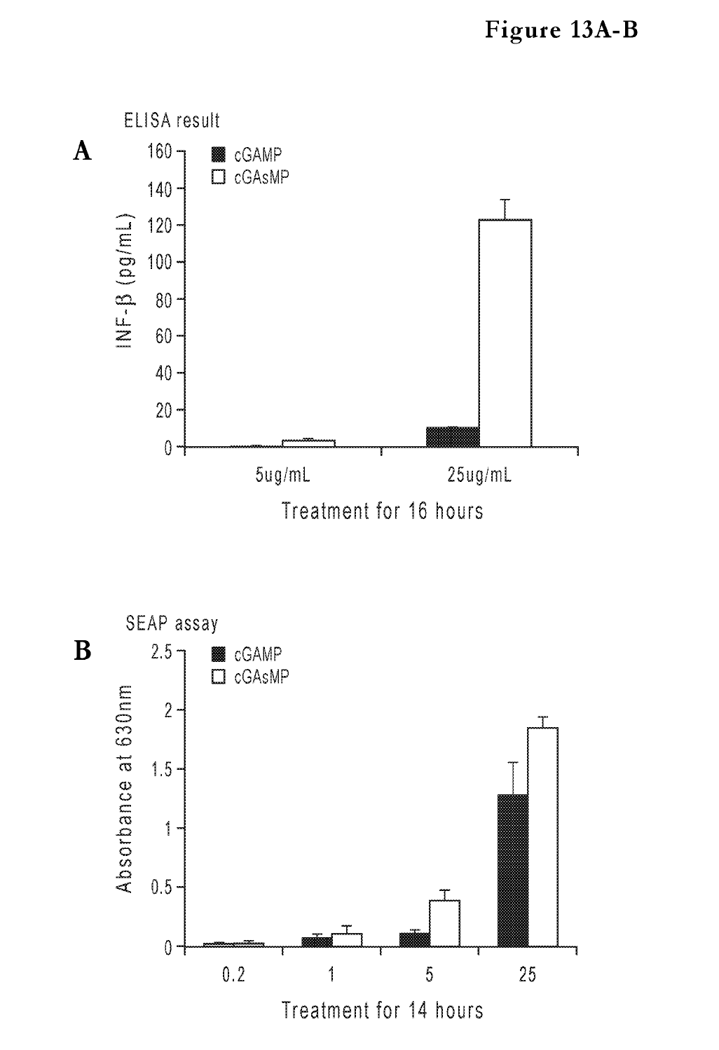

FIGS. 13A-B show that both cGAMP and cGAsMP can induce beta production in THP1 cells, but that cGAsMP, the phosphorothioate derivative of cGAMP, has enhanced potency. IFN-β ELISA of THP1 cells treated with 5 and 25 μg/ml of cGAMP and cGAsMP shows that cGAsMP can induce 5-10 times higher levels of IFN-β (FIG. 13A). Consistent with these results, we also observed that cGAsMP is more potent than cGAMP in inducing the expression of a IFN-β reporter gene in THP1 cells treated with 0.2 to 25 μg/ml of cGAMP and cGAsMP (FIG. 13B).

### Example 9

### Antitumor Activities of cGAMP and cGAsMP

An MTT assay was used to show that both cGAMP and cGAsMP have anticancer activity.
A. Reagents Used in MTT Assay

MTT solution: 5 mg/mL Thiazolyl Blue Tetrazolium Bromide (MTT) in PBS. The solution was filter sterilized after adding MTT and stored at −20° C.; MTT solvent: 4 mM HCl, 0.1% Nondet P-40 (NP40) in isopropanol. cGAMP or cGAsMP solutions: 10-30 mg/ml in PBS, filter sterilized using a 0.2 μm filter.
B. MTT Assay for Attaching Cancer Cell Lines SF539, U251, A498, and ACHN

On day one, one T-25 flask was trypsinized and 5 ml of complete media was added to the cells. The cells and media were centrifuged in a sterile 15 ml falcon tube at 300×g rcf in the swinging bucket rotor for 5 min. Media was removed and cells resuspended in 1.0 ml complete RPMI 1640 media. Cells were counted and recorded per ml. Cells were diluted (cv=cv) to 75,000 cells per ml with complete RPMI media. 100 μl of cells (7500 total cells) were added into each well of a 96 well plate and incubated overnight. 24 hours later, 100 μl of medium or cGAMP or cGAsMP solutions were added to each well. On the fifth day, 20 μl of 5 mg/ml MTT were added to each well. One set of wells with MTT but no cells served as a control. All steps were done aseptically. The wells were incubated for 3.5 hours at 37° C. in a CO₂ incubator. Media was carefully removed, taking care not to disturb the cells. No PBS rinse was performed. 150 μl MTT solvent was added. The plate was covered with foil and cells agitated on orbital shaker for 15 min. The absorbance was measured at 590 nm using a plate reader. Each assay was repeated five times.
C. MTT Assay for Non-Attaching Cancer Cell Lines SR or CCRF-CEM

Cells were centrifuged in a sterile 15 ml falcon tube at 300×g rcf in the swinging bucked rotor for 5 min. Media was removed and cells resuspended with 1.0 ml complete RPMI 1640 media. Cells were counted and recorded per ml. The cells were diluted (cv=cv) to 100,000 cells per ml using complete media. 100 μl of cells were added (10000 total cells) into each well of a 96 well plate and incubated overnight. 24 hours later, 100 μl of medium or cGAMP or cGAsMP solutions were added to each well. On the fifth day, 20 μl of 5 mg/ml MTT were added to each well. One set of wells with MTT but no cells served as control. Wells were incubated for 3.5 hours at 37° C. in a CO₂ incubator. 150 μl media was removed from each well, taking care not to disturb cells. No PBS rinse was performed. 150 μl MTT solvent was added. Only when necessary, pipetting up and down was required to completely dissolve the MTT formazan crystals. The plate was covered with foil and cells agitated on orbital shaker for 15 min. The absorbance was measured at 590 nm using a plate reader. Each assay was repeated five times.

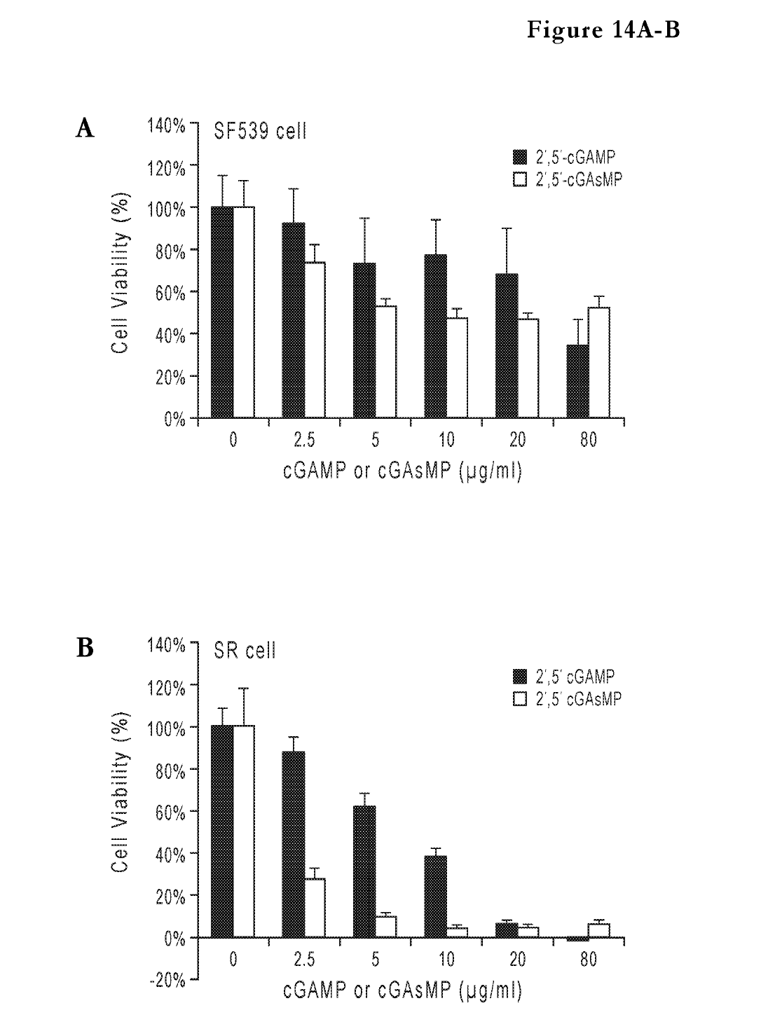

FIG. 14A shows the results in an MTT of treatment of a neuronal cancer cell line SF539 treated with cGAMP and cGAsMP. FIG. 14B shows the results in an MTT assay of a leukemia cell line SR treated with cGAMP and cGAsMP. The figures demonstrate that both cGAMP and cGAsMP have antitumor activity in the neuronal and leukemia cell lines evaluated and that cGAsMP has generally a bigger impact on cell viability at lower concentrations.

### Example 10

### cGAMP Represses Tumor Growth In Vivo

A. In Vivo Assessment of Colon Cancer Model

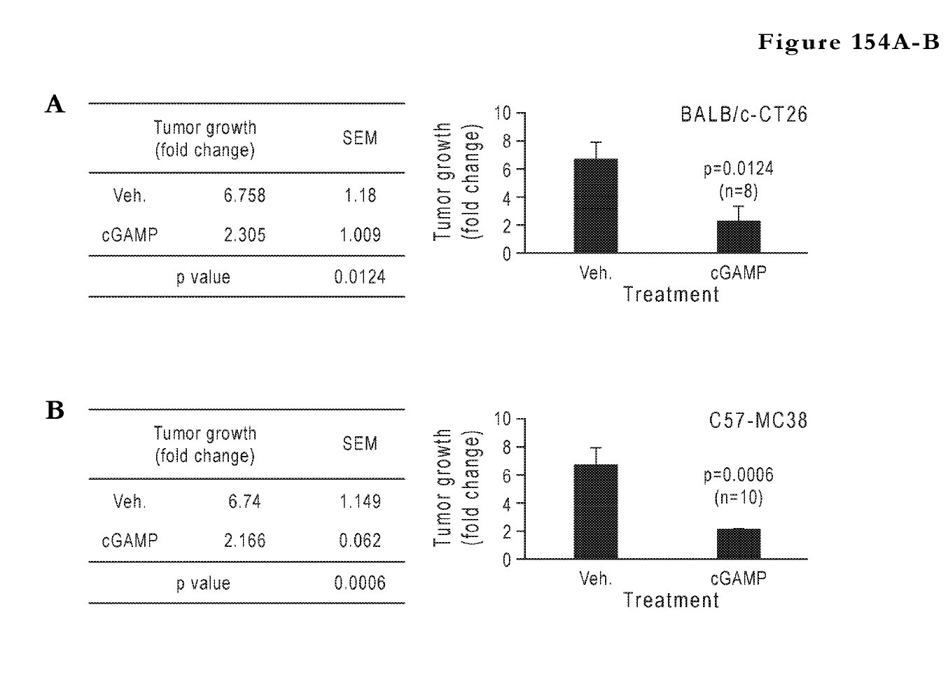

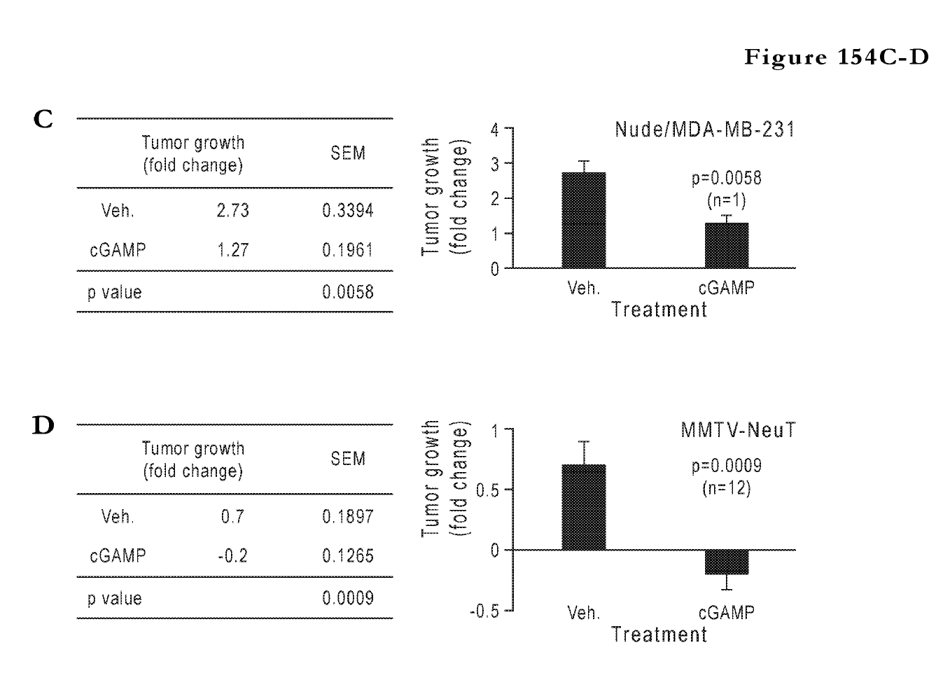

Colon cancer CT26 and MC38 cells were implanted by subcutaneous injection in two flanks of 5-6-week-old BALB/c and C57B/J mice, respectively. Treatment began at day 14 after implantation of the colon cancer cells and mice with tumor sizes from 100-200 mm³ were treated. cGAMP was administered through intratumor injection at a concentration of 4 mg/kg once a day for three consecutive days. After the treatment phase, tumor growth was measured for 7 days and the fold change in tumor size was determined every other day. Result from day 7 post-treatment are shown in FIG. 15A (colon cancer CT26 cells implanted in BALB/c mice) and FIG. 15B (colon cancer MC38 cells implanted into C57B/J mice). In vivo results show that cGAMP administration is effective in reducing tumor growth.
B. In Vivo Assessment of Breast Cancer Model

Breast cancer MDA-MB-231 cells were implanted by subcutaneous injection in two flanks of 5-6-week-old BALB/c nu/nu mice. The tumor growth was monitored for 14 days and growth rate was examined using serial caliper measurements. The tumor volume was calculated using the equation (a×b²)/2 where “a” and “b” are length and width of the tumor, respectively. Treatment began at day 14 after implantation of the breast cancer cells. When tumor grew to 100-200 mm³, cGAMP was administered at a concentration of 10 mg/kg for seven consecutive days. After the treatment phase, tumor growth was measured for 7 days and the fold change in tumor size was determined every other day. Results from day 7 post-treatment are shown in FIG. 15C. In vivo results show that cGAMP administration is effective in reducing tumor growth, with a p value of 0.0058.
C. In Vivo Assessment of Breast Cancer Model

The MMTV-BALB-neuT mouse constitutes an aggressive model of rat her-2/neu mammary carcinogenesis, providing an effective model for spontaneous breast cancer. These mice express unactivated neu under the transcriptional control of the mouse mammary tumor virus promoter/enhancer. When tumor reached 200 mm³ at around 8 months, mice were grouped based on tumor size. cGAMP was administered at a concentration of 0.1 mg per mouse through intra-tumor injection once a day for three consecutive days. Comparisons were made between vehicle (veh.) and cGAMP treatment. The tumor growth was monitored for 4 days and growth rate was examined using serial caliper measurements. The tumor volume was calculated using the equation (a×b²)/2 where “a” and “b” are length and width of the tumor, respectively. At the completion of the experiments, tumors were excised and statistical significance of differences in tumor volume was analyzed. Results from day 4 post-treatment are shown in FIG. 15D. These in vivo results show that cGAMP administration is effective for both reducing tumor growth and reducing tumor size, with a p value of 0.0009.

### Example 11

### Additional Embodiments

Additional embodiments may be found in the following numbered items.

Item 1. A method of treating cancer in a patient comprising administering cGAMP or cGAsMP to a patient having cancer and allowing the cGAMP or cGAsMP to treat the cancer.

Item 2. A method of inhibiting growth of cancer cells comprising
a. providing a population of cancer cells;
b. exposing the cancer cells to cGAMP or cGAsMP; and
c. allowing the cGAMP or cGAsMP to inhibit the growth of the cancer cells.

Item 3. The method of any one of items 1-2, wherein STING expression level in the cancer is at least about 1, 1.2, 1.25, 1.3, 1.4, 1.5, 1.6, 1.7, 1.8, 1.9, 2.0, 2.25, 2.5, 2.75, 3.0, 3.25, 3.5, 3.75, 4.0, 4.25, or 4.5 fold higher than an average level in normal cells.

Item 4. The method of any one of items 1-3, wherein cGAS expression level are within the lower 50%, 45%, 40%, 35%, 30%, 25%, 20%, 15%, or 10% of patients, when evaluating the cGAS level in a pool of patients.

Item 5. The method of any one of items 1-4, wherein the pool of patients has only cancer patients.

Item 6. The method of any one of items 1-5, wherein the pool of patients has both cancer patients and normal patients.

Item 7. The method of any one of items 1-6, wherein the cancer is CNS cancer, renal cancer, or lymphoma.

Item 8. The method of item 7, wherein the CNS cancer is glioblastoma.

Item 9. The method of item 7, wherein the renal cancer is a renal carcinoma.

Item 10. The method of any one of items 1-6, wherein the cancer is leukemia (including, but not limited to, acute myeloid leukemia, chronic myelogenous leukemia, and pro-B acute lymphoblastic leukemia), lymphoma (including, but not limited to, activated B-cell-like diffuse large B-cell lymphoma, diffuse large B-cell lymphoma, follicular lymphoma, anaplastic large cell lymphoma, angioimmunoblastic T-cell lymphoma, ALK-positive, Burkitt's lymphoma, Hodgkin's lymphoma, nodular lymphocyte predominant Hodgkin's lymphoma, T-cell/histocyte-rich large B-cell lymphoma, and germinal center B-cell-like diffuse large B-cell lymphoma), gastric cancer (diffuse gastric adenocarcinoma, gastric intestinal type adenocarcinoma, and gastric mixed adenocarcinoma), esophageal cancer (Barrett's esophagus, esophageal squamous cell carcinoma, and esophageal adenocarcinoma), colorectal cancer, pancreatic cancer, embryonal carcinoma, mixed germ cell tumor, seminoma, teratoma, yolk sac tumor, testicular teratoma, thyroid cancer, renal carcinoma, melanoma, glioblastoma, tongue carcinoma, breast cancer, oral cavity carcinoma, oropharyngeal carcinoma, and tonsillar carcinoma.

Item 11. The method of any one of items 1-10, wherein the cancer cells are screened ex vivo to determine whether cGAMP or cGAsMP will inhibit growth of the cancer cells.

Item 12. The method of any one of items 1-11, wherein the cancer cells are screened ex vivo to determine whether cGAMP or cGAsMP will induce the expression of IFN-β before the cGAMP or cGAsMP is administered to the patient.

Item 13. The method of any one of items 1-12, wherein the method comprises administering 0.1 to 1 mg/kg cGAMP or cGAsMP to the patient.

Item 14. A method for enzymatically synthesizing cGAMP or cGAsMP comprising:
a. providing recombinant cGAS; and
b. combining cGAS with ATP or ATP phosphorothioate, GTP, and dsDNA to synthesize cGAMP or cGAsMP.

Item 15. The method of item 14, wherein modified nucleotides are used in the synthesis method.

Items 16. The method of any one of items 14-15, wherein the synthesis may be conducted in a single pot.

Item 17. The method of any one of items 14-16, wherein the synthesis may be conducted in a single step.

Item 18. A method for purifying cGAMP or cGAsMP comprising:
a. providing a mixture of cGAMP or cGAsMP and at least one other compound chosen from dsDNA and cGAS;
b. separating cGAMP or cGAsMP from dsDNA and cGAS by ultrafiltration;
c. purifying cGAMP or cGAsMP using ion exchange chromatography; and
d. removing salt from cGAMP or cGAsMP by lyophilization.

Item 19. A method for enzymatically synthesizing and purifying cGAMP or cGAsMP comprising:
a. providing recombinant cGAS;
b. combining cGAS with ATP or ATP phosphorothioate, GTP, and dsDNA to synthesize cGAMP;
c. separating cGAMP or cGAsMP from dsDNA and cGAS by ultrafiltration;
d. purifying cGAMP or cGAsMP using ion exchange chromatography and optionally gel filtration chromatography; and
e. removing salt from cGAMP or cGAsMP by lyophilization.

Item 20. The method of any one of items 14-17 or 19, wherein cGAS is combined with ATP or ATP phosphorothioate and GTP in the presence of an ingredient to reduce nonspecific interactions.

Item 21. The method of item 20, wherein the ingredient to reduce nonspecific interactions is salmon sperm DNA.

Item 22. The method of any one of items 14-17 or 19-21, wherein cGAS is combined with ATP and GTP in the presence of at least one buffer, salt, and/or antioxidant.

Item 23. The method of item 22, wherein at least one buffer is HEPES buffer.

Item 24. The method of any one of items 22-23, wherein at least one salt is MgCl₂ and/or NaCl.

Item 25. The method of any one of items 22-24, wherein at least one antioxidant is β-mercaptoethanol.

Item 26. The method of any one of items 18-25, wherein the precipitant was removed by centrifugation at 4000×g for 15 minutes.

Item 27. The method of any one of items 18-26, wherein the ultrafiltration occurs through an ultrafiltration filter with a 10 kD pore size.

Item 28. The method of any one of items 18-27, wherein the ion exchange chromatography is on a Q Sepharose column

Item 29. The method of any one of item 18-28, wherein the Q Sepharose column is eluted with a volatile salt buffer containing ammonium acetate.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). In some instances, the term about may include numerical values that are rounded to the nearest significant figure.

What is claimed is:

1. A method of treating cancer in a patient comprising administering cGAMP or cGAsMP to a patient having cancer and allowing the cGAMP or cGAsMP to treat the cancer.

2. A method of inhibiting growth of cancer cells comprising
a. providing a population of cancer cells;
b. exposing the cancer cells to cGAMP or cGAsMP; and
c. allowing the cGAMP or cGAsMP to inhibit the growth of the cancer cells.

3. The method of claim 1, wherein STING expression level in the cancer is at least about 1, 1.2, 1.25, 1.3, 1.4, 1.5, 1.6, 1.7, 1.8, 1.9, 2.0, 2.25, 2.5, 2.75, 3.0, 3.25, 3.5, 3.75, 4.0, 4.25, or 4.5 fold higher than an average level in normal cells.

4. The method of claim 1, wherein cGAS expression level are within the lower 50%, 45%, 40%, 35%, 30%, 25%, 20%, 15%, or 10% of patients, when evaluating the cGAS level in a pool of patients.

5. The method of claim 1, wherein the cancer is CNS cancer, renal cancer, or lymphoma.

6. The method of claim 1, wherein the cancer is leukemia (including, but not limited to, acute myeloid leukemia, chronic myelogenous leukemia, and pro-B acute lymphoblastic leukemia), lymphoma (including, but not limited to, activated B-cell-like diffuse large B-cell lymphoma, diffuse large B-cell lymphoma, follicular lymphoma, anaplastic large cell lymphoma, angioimmunoblastic T-cell lymphoma, ALK-positive, Burkitt's lymphoma, Hodgkin's lymphoma, nodular lymphocyte predominant Hodgkin's lymphoma, T-cell/histocyte-rich large B-cell lymphoma, and germinal center B-cell-like diffuse large B-cell lymphoma), gastric cancer (diffuse gastric adenocarcinoma, gastric intestinal type adenocarcinoma, and gastric mixed adenocarcinoma), esophageal cancer (Barrett's esophagus, esophageal squamous cell carcinoma, and esophageal adenocarcinoma), colorectal cancer, pancreatic cancer, embryonal carcinoma, mixed germ cell tumor, seminoma, teratoma, yolk sac tumor, testicular teratoma, thyroid cancer, renal carcinoma, melanoma, glioblastoma, tongue carcinoma, breast cancer, oral cavity carcinoma, oropharyngeal carcinoma, and tonsillar carcinoma.

7. The method of claim 1, wherein the method comprises administering 0.1 to 1 mg/kg cGAMP or cGAsMP to the patient.

8. A method for enzymatically synthesizing cGAMP or cGAsMP comprising:
a. providing recombinant cGAS; and
b. combining cGAS with ATP or ATP phosphorothioate, GTP, and dsDNA to synthesize cGAMP or cGAsMP.

9. A method for purifying cGAMP or cGAsMP comprising:
a. providing a mixture of cGAMP or cGAsMP and at least one other compound chosen from dsDNA and cGAS;
b. separating cGAMP or cGAsMP from dsDNA and cGAS by ultrafiltration;
c. purifying cGAMP or cGAsMP using ion exchange chromatography; and
d. removing salt from cGAMP or cGAsMP by lyophilization.

10. A method for enzymatically synthesizing and purifying cGAMP or cGAsMP comprising:
a. providing recombinant cGAS;
b. combining cGAS with ATP or ATP phosphorothioate, GTP, and dsDNA to synthesize cGAMP;
c. separating cGAMP or cGAsMP from dsDNA and cGAS by ultrafiltration;
d. purifying cGAMP or cGAsMP using ion exchange chromatography and optionally gel filtration chromatography; and
e. removing salt from cGAMP or cGAsMP by lyophilization.

11. The method of claim 10, wherein cGAS is combined with ATP or ATP phosphorothioate and GTP in the presence of an ingredient to reduce nonspecific interactions.

12. The method of claim 11, wherein the ingredient to reduce nonspecific interactions is salmon sperm DNA.

13. The method of claim 10, wherein cGAS is combined with ATP and GTP in the presence of at least one buffer, salt, and/or antioxidant.

14. The method of claim 13, wherein at least one buffer is HEPES buffer.

15. The method of claim 13, wherein at least one salt is MgCl₂ and/or NaCl.

16. The method claim 10, wherein at least one antioxidant is β-mercaptoethanol.

17. The method claim 10, wherein precipitant was removed by centrifugation at 4000×g for 15 minutes.

18. The method of claim 10, wherein the ultrafiltration occurs through an ultrafiltration filter with a 10 kD pore size.

19. The method of claim 10, wherein the ion exchange chromatography is on a Q Sepharose column.

20. The method of claim 19, wherein the Q Sepharose column is eluted with a volatile salt buffer containing ammonium acetate.
